(19) United States
(12) Patent Application Publication
(10) Pub. No.: US 2025/0161483 A1
(43) Pub. Date: May 22, 2025

(54) METHODS AND PHARMACEUTICAL COMPOSITIONS FOR THE TREATMENT AND THE PREVENTION OF CARDIOMYOPATHY ASSOCIATED WITH FRIEDREICH ATAXIA

(71) Applicants: LEXEO Therapeutics, Inc., New York, NY (US); Cornell University, Ithaca, NY (US)

(72) Inventors: Ronald G. CRYSTAL, New York, NY (US); Stephen M. KAMINSKY, Bronx, NY (US); Dolan SONDHI, New York, NY (US); Jay A. BARTH, Teaneck, NJ (US); Richie KHANNA, Somerset, NJ (US)

(21) Appl. No.: 18/834,836

(22) PCT Filed: Feb. 1, 2023
(86) PCT No.: PCT/US2023/061766

§ 371 Date: Jul. 31, 2024

Related U.S. Application Data
(60) Provisional application No. 63/341,669, filed on May 13, 2022.
(60) Provisional application No. 63/305,494, filed on Feb. 1, 2022.

(51) Int. Cl. A61K 48/00; A61K 31/573; A61K 38/17; A61P 25/28

(52) U.S. Cl. CPC A61K 48/0033; A61K 31/573; A61K 38/1709; A61P 25/28

(57) ABSTRACT

The present disclosure provides methods and compositions for the treatment of cardiomyopathy associated with Friedreich ataxia. The methods and compositions of the present disclosure comprise AAV vectors and AAV viral vectors comprising transgene nucleic acid molecules comprising nucleic acid sequences encoding for a frataxin polypeptide.

### CROSS-REFERENCE TO RELATED APPLICATIONS

This application claims priority to, and the benefit of, U.S. Provisional Application No. 63/305,494 filed Feb. 1, 2022 and U.S. Provisional Application No. 63/341,669 filed May 13, 2022. The contents of each of which is hereby incorporated by reference in their entireties.

### GOVERNMENT FUNDING

This invention was made with government support under R61 HL151355 awarded by the National Institute of Health. The government has certain rights in the invention.

### SUBMISSION OF SEQUENCE LISTING ON ASCII TEXT FILE

The contents of the electronic sequence listing (LEXE_009_001WO_SeqList_ST26.xml; Size: 17,703 bytes; and Date of Creation: Jan. 31, 2023) are herein incorporated by reference in its entirety.

### FIELD OF THE INVENTION

The invention relates to a method for preventing or treating a cardiomyopathy associated with Friedreich ataxia in a subject in need thereof, comprising administering to said subject a therapeutically effective amount of an AAV vector which comprises a frataxin (FXN) encoding nucleic acid.

### BACKGROUND OF THE INVENTION

Friedreich's ataxia is an autosomal recessive, progressive, neurodegenerative movement disorder with a typical age of onset between 10 and 15 years. Clinical presentation includes unsteady gait and frequent falling, with a progressive impact on mobility and coordination.

Friedreich's ataxia is caused by mutations in the autosomal FXN gene on 9q214. The FXN protein is directed to the mitochondria (Lupoli 2018; FEBS Lett, 592:718-27). Ninety-six percent (96%) of FA patients are homozygous for a GAA trinucleotide expansion within the first intron, which causes transcriptional silencing through a mechanism involving epigenetic changes, leading ultimately to a reduction in the levels of the FXN messenger RNA (mRNA) compared to normal healthy subjects. Affected individuals have a reduction of the FXN protein in all tissues (Puccio 2000; Hum Mol Genet, 9:887-92). The remaining subjects (4%) are compound heterozygous for a GAA expansion and a more classical mutation (point mutation, small deletion or insertion) on the other allele leading to loss of function of the FXN protein (Campuzano 1996; Science, 271: 1423-7).

The FXN gene is composed of seven exons spread over 85 kb of genomic DNA (Pandolfo 2006; Genetic Instabilities and Neurological Diseases: Chapter 17). The major transcript (1.3 kb) is composed of the first 5 exons, localized within a 40 kb interval (Campuzano 1996; Pandolfo 2006). Frataxin encodes a 210-amino acid protein which undergoes a maturation process, typical of nuclear-encoded mitochondrial proteins (Miranda 2002; FEBS Lett, 512: 291-7; Puccio 2001; Nat Genet, 27: 181-6). The targeting sequence is contained between amino acids 1-80, consisting of positively charged residues (arginines) in an alpha-helix (Martelli 2014; Front Pharmacol, 5: 130). The maturation process occurs in 2 steps by the mitochondrial processing peptidase, a cleavage between positions 41 and 42 leading to the intermediate form of FXN, followed by cleavage resulting in the mature form starting at amino acid 818 (Seznec 2004; Hum Mol Genet. 2004; 13:1017-1024).

Reduced levels of FXN result in mitochondrial dysfunction, with loss of iron-sulfur (Fe—S) cluster enzyme activities (aconitase and respiratory chain complexes I-III), mitochondrial iron accumulation and increased sensitivity to oxidative stress (Adinolfi 2009; Nat Struct Mol Biol, 16: 390-6; Huynen 2001; Hum Mol Genet, 10: 2463-8; Muhlenhoff 2002; J Biol Chem, 277: 29810-6; Puccio 2001; Schmucker 2011; PLOS One, 6: e16199; Tsai 2010; Biochemistry, 49: 9132-9).

Disclosed is an AAV vector encoding the human FXN gene. The AAV vector is infused intravenously. The rh 10 capsid is designed specifically to target delivery to myocardium where it delivers a normal copy of the FXN gene, leading to increased expression of FXN in cardiomyocytes. The vector is hypothesized to stabilize and/or improve cardiomyopathy associated with FA, as measured by cardiopulmonary exercise testing (CPET) and cardiac magnetic resonance imaging (MRI).

### SUMMARY OF THE INVENTION

The disclosure provides a method of treating or preventing cardiomyopathy associated with Friedreich ataxia in a subject, the method comprising administering to the subject a therapeutically effective amount of an adeno associated virus (AAV) vector which comprises a nucleic acid sequence encoding a frataxin (FXN) polypeptide or a fragment thereof, wherein the vector is administered to the subject intravenously at a dose ranging from about 1.0×10¹⁰ gc/kg to about 6.0×10¹⁴ gc/kg.

The disclosure provides, a method of treating or preventing cardiomyopathy associated with Friedreich ataxia in a subject, the method comprising administering to the subject a therapeutically effective amount of an adeno associated virus (AAV) vector which comprises a nucleic acid sequence encoding a frataxin (FXN) polypeptide or fragment thereof, wherein the AAV vector comprises the nucleic acid sequence set forth in SEQ ID NO: 9, wherein the vector is administered the subject intravenously at a dose of about 1.8×10¹¹ gc/kg or about 5.6×10¹¹ gc/kg.

In some aspects, the AAV vector comprises in the 5′ to 3′ direction: a first AAV ITR sequence; an enhancer sequence; a promoter sequence; a chimeric intron; the nucleic acid sequence encoding a frataxin (FXN) polypeptide; a polyA sequence; and a second ITR sequence.

In some aspects, the nucleic acid sequence encoding a frataxin (FXN) polypeptide comprises the nucleic acid sequence set forth in SEQ ID NO: 3. In some aspects, the first ITR sequence comprises the nucleic acid sequence set forth in SEQ ID NO: 1. In some aspects, the second ITR sequence comprises the nucleic acid sequence set forth in SEQ ID NO: 2. In some aspects, the enhancer sequence comprises the nucleic acid sequence set forth in SEQ ID NO: 5. In some aspects, the promoter sequence comprises the nucleic acid sequence set forth in SEQ ID NO: 6. In some aspects, the polyA sequence comprises the nucleic acid sequence set forth in SEQ ID NO: 8. In some aspects, the AAV vector comprises the nucleic acid sequence set forth in SEQ ID NO: 9.

In some aspects, the AAV vector is packaged as an AAV viral vector comprising an AAV capsid protein. In some aspects, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some aspects, the AAV capsid protein is an AAVrh10 capsid protein.

In some aspects, the dosage is about 1.8×10¹¹ gc/kg. In some aspects, the dosage is about 5.6×10¹¹ gc/kg.

In some aspects, the subject is further administered prednisone. In some aspects, the prednisone is administered at a dosage of: 40 mg, once daily 24 hours prior to AAV viral vector administration: 40 mg once daily for week 1 through week 8 post-AAV viral vector administration: 30 mg once daily for week 9 post-AAV viral vector administration: 20 mg once daily for week 10 post-AAV viral vector administration: 10 mg once daily for week 11 post-AAV viral vector administration: 5 mg once daily for week 12 post-AAV viral vector administration: 2.5 mg once daily for week 13 post-AAV viral vector administration; and 2.5 mg every other day for week 14 post-AAV viral vector administration.

In some aspects, the intravenous administration occurs over about 60 minutes.

In some aspects, the subject experiences an increase in peak VO2 following AAV vector administration relative to a pre-AAV vector administration baseline.

In some aspects, the peak VO2 in the subject is measured about, 12 weeks, about 24 weeks, about 36 weeks, about 52 weeks, about 18 months, about 2 years, about 3 years, about 4 years, and/or about 5 years post-AAV vector administration. In some aspects, peak VO2 is measured by cardiopulmonary exercise testing (CPET) using arm ergometry.

In some aspects, the subject experiences a decrease in Left Ventricular Mass index (LVMi) following AAV vector administration relative to a pre-AAV vector administration baseline. In some aspects, LVMi is measured about 12 weeks, about 24 weeks, about 36 weeks, about 52 weeks post-AAV vector administration, about 18 months, about 2 years, about 3 years, about 4 years, and/or about 5 years. In some aspects, LVMi is measured by cardiac MRI.

In some aspects, the subject, following AAV vector administration, experiences one or more of decreased global longitudinal strain, increased stroke volume, increased left ventricular ejection fraction (LVEF), and decreased or stable cardiac fibrosis as measured by cardiac MRI relative to a pre-AAV vector administration baseline. In some aspects, the measurement by cardiac MRI occurs about 12 weeks, about 24 weeks, about 36 weeks, about 52 weeks, about 18 months, about 2 years, about 3 years, about 4 years, and/or about 5 years post-AAV vector administration.

In some aspects, the subject, following AAV vector administration, experiences decreased serum NTproBNP, hsTNT, and CK-MB levels relative to a pre-AAV vector administration baseline.

In some aspects, the subject, following AAV vector administration, experiences decreased cardiac arrythmias. In some aspects, cardiac arrythmias are evaluated by remote cardiac rhythm monitoring.

In some aspects, the subject, following AAV vector administration, experiences decreased fatigue as measured by the Modified Fatigue Impact Scale (MFIS) or Fatigue Severity Scale (FSS) relative to a pre-AAV vector administration baseline.

In some aspects, the subject, following AAV vector administration, experiences improvements in exertional symptoms (during CPET via arm ergometry) as measured by the Modified Borg Dyspnea, Borg Rating of Perceived Exhaustion (RPE), and the angina scale using CPET relative to a pre-AAV vector administration baseline.

In some aspects, the subject, following AAV vector administration, experiences increased FXN expression levels relative to a pre-AAV vector administration baseline. In some aspects, the FXN levels are myocardial FXN expression levels. In some aspects, myocardial FXN expression is measured by cardiac biopsy.

In some aspects, the subject experiences an at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 150%, at least about 200%, at least about 300%, at least about 400% increase in FXN expression levels.

In some aspects, the subject, following AAV vector administration, experiences improvements or stabilizations in the Friedreich's Ataxia Rating Scale (FARS) or modified Friedreich's Ataxia Rating Scale (mFARS) relative to a pre-AAV vector administration baseline.

In some aspects, the subject, following AAV vector administration, experiences improvements or stabilizations in the Scale for Assessment and Rating of Ataxia (SARA) relative to a pre-AAV vector administration baseline.

In some aspects, the subject, following AAV vector administration, experiences improvements or stabilizations in the Shortness of Breath-Daily Activities Score (SOBDA) relative to a pre-AAV vector administration baseline.

In some aspects, the subject, following AAV vector administration, experiences improvements or stabilizations in the Seattle Angina Questionnaire (SAQ) relative to a pre-AAV vector administration baseline.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

FIGS. 1A-B show two graphs depicting the impact of varying doses of AAVrh.10FXN on cardiac expression of hFXN in MCK mice. FIG. 1A shows a line graph depicting dose-dependent cardiac hFXN levels following intravenous administration of three dosages. The x-axis depicts administered dosage. The y-axis depicts total cardiac levels of hFXN protein. FIG. 1B shows a bar graph depicting the dose-dependent cardiac hFXN levels relevant to estimated human levels to convert a FA homozygote to a heterozygote. The x-axis depicts dosage. The y-axis depicts hFXN protein levels in cardiac tissue. The range of heart frataxin levels in subjects heterozygous for FA is depicted as a gray box within the graph.

FIGS. 2A-B show two graphs depicting the efficacy of intravenous administration of AAVrh.10hFXN on body weight of male and female MCK mice. The x-axis depicts time after vector administration in days. The y-axis depicts body weight in grams. The expected death of PBS-treated MCK mice is depicted by a vertical, dashed-line. FIG. 2A shows male mice. FIG. 2B shows female mice.

FIG. 3 shows a series of echocardiography images taken of mice cardiac tissue across three treatment groups (WT/untreated; MCK/PBS mock-treated; MCK/1.8×10¹² gc/kg AAVrh.10hFXN) at multiple time points post-treatment. Images depict the lateral (top) and septal (bottom) walls of the left ventricle from parasternal short axis view. The vertical dashed lines measure the relative position of the walls during systole (green) and diastole (red).

FIGS. 4A-B show two graphs depicting improved cardiac function in MCK mice treated with 1.8×10¹² gc/kg AAVrh.10hFXN via intravenous administration. FIG. 4A shows cardiac ejection fraction in mice in three treatment groups. Lines are labeled by treatment group. The x-axis depicts mice age in weeks. The y-axis depicts ejection fraction percentage. FIG. 4B shows cardiac fractional shortening in three groups of mice as indicated next to the respective lines. The x-axis depicts mice age in weeks. The y-axis depicts fractional shortening percentage.

FIG. 5 shows a survival curve depicting dose-dependent impact of intravenous administration of AAVrh.10hFXN on MCK mouse survival. Mice received three doses of AAVrh.10hFXN, or mock-treatment with PBS. Lines are labeled according to treatment. The x-axis depicts the age of mice in days. The y-axis depicts the percent survival.

FIG. 6 shows a schematic of an exemplary model of assessing the impact in nonhuman primates (NHPs) of the minimally effective and significantly effective doses as determined in MCK mice. Two doses were administered intravenously and compared to mock-treated PBS control. Heart tissue was collected 12 weeks post-administration and hFXN levels evaluated.

FIGS. 7A-B show two graphs depicting the impact of AAVrh.10hFXN on cardiac expression of hFXN in nonhuman primates using the exemplary model depicted in FIG. 6. FIG. 7A shows a bar graph depicting the dose-dependent hFXN levels in nonhuman primate cardiac tissue. The x-axis depicts treatment group. The y-axis depicts hFXN levels. hFXN levels in the PBS control group are indicated by a horizontal dashed line. FIG. 7B shows a bar graph depicting hFXN relevance to heterozygote target levels. The x-axis depicts vector dose. The y-axis depicts hFXN levels. The range of heart frataxin levels in subjects heterozygous for FA is depicted by a gray box in the graph.

FIGS. 8A-8C depict an expanded dosage study in MCK mice of AAVrh.10hFXN. FIG. 8A is a table depicting the dosages of AAVrh.10hFXN administered to mice. FIG. 8B is a graph depicting the expression of human frataxin in the liver of treated mice following administration of AAVrh.10hFXN a the indicated dosages. FIG. 8C is a graph depicting the expression of human frataxin in the heart of treated mice following administration of AAVrh.10hFXN at the indicated dosages.

### DETAILED DESCRIPTION OF THE INVENTION

The disclosure provides a method of treating or preventing cardiomyopathy associated with Friedreich ataxia in a subject. In some aspects, the method comprises administering to the subject a therapeutically effective amount of an adeno associated virus (AAV) vector which comprises a nucleic acid sequence encoding a frataxin (FXN) polypeptide or a fragment thereof. In some aspects, the vector is administered in a therapeutically effective amount at a dose ranging from 1.0×10¹⁰ gc/kg to about 6.0×10¹⁴ gc/kg. In some aspects, the vector is administered intravenously.

### AAV Vectors

In some aspects, an isolated nucleic acid sequence comprising the nucleic acid sequence encoding frataxin (FXN) can be an AAV vector.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPHP.B, AAVrh74 or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV11, AAV12, AAV13, AAVPHP.B, AAVrh74 and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4 (22):e131610) and AAV-NP59 (described in detail in Paulk et al. Molecular Therapy, 2018; 26 (1): 289-303).

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077: the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716: the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh. 74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158:97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958 June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat Acad Sc USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule comprising a polynucleotide sequencing encoding for at least one short hairpin RNA (shRNA) molecules directed against UBE3A-ATS and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22 (11): 1900-1909 (2014). The nucleotide sequences of the genomes of various AAV serotypes are known in the art.

An “AAV vector” as used herein is in reference to a vector comprising, consisting essentially of, or consisting of one or more transgene sequences and one or more AAV inverted terminal repeat sequences (ITRs). In some aspects, AAV vectors contain one or more of an enhancer, a promoter, at least one nucleic acid that may encode at least one protein, an intronic sequence, and a polyA sequence.

In some aspects, the invention relates to an AAV vector which comprises a frataxin (FXN) encoding nucleic acid for use in the treatment or prevention of a cardiomyopathy in a subject in need thereof.

In some aspects, the invention relates to an AAV vector which comprises a frataxin (FXN) encoding nucleic acid for use in the treatment or prevention of a cardiomyopathy associated with Friedreich ataxia in a subject in need thereof.

In some aspects, the invention relates to an AAV vector which comprises a frataxin (FXN) encoding nucleic acid for reversing or stabilizing symptoms of cardiomyopathy associated with Friedreich ataxia in a subject in need thereof.

In some aspects, the invention relates to an AAV vector which comprises a frataxin (FXN) encoding nucleic acid for reversing the dysfunction of cardiac mitochondria associated with Friedreich ataxia in a subject in need thereof.

In some aspects, the invention relates to an AAV vector which comprises a frataxin (FXN) encoding nucleic acid for improving the cardiac mitochondria associated with Friedreich ataxia in a subject in need thereof.

In some aspects, the invention relates to an AAV vector which comprises a frataxin (FXN) encoding nucleic acid for restoring cardiac function in a subject suffering of a cardiomyopathy associated with Friedreich ataxia.

In some aspects, the invention relates to an AAV vector which comprises a frataxin (FXN) encoding nucleic acid for improving cardiac function in a subject suffering of a cardiomyopathy associated with Friedreich ataxia.

In some aspects, the invention relates to an AAV vector which comprises a frataxin (FXN) encoding nucleic acid for use in the treatment a cardiomyopathy associated with Friedreich ataxia in an asymptomatic or pre-symptomatic subject in need thereof.

In some aspects, the invention relates to an AAV vector which comprises a frataxin (FXN) encoding nucleic acid for use in the treatment a cardiomyopathy associated with Friedreich ataxia in a symptomatic subject in need thereof.

In some aspects, AAV vectors used in methods of the disclosure comprise in the 5′ to 3′ direction a first AAV ITR sequence; an enhancer sequence, a promoter sequence; a chimeric intron sequence, a nucleic acid sequence encoding frataxin; a polyA sequence; and a second ITR sequence.

In some aspects, a frataxin-encoding AAV vector of the disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 9.

### Nucleic Acid Sequences Encoding Frataxin

In some aspects, a nucleic acid sequence encoding a frataxin (FXN) polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 3.

In some aspects, the frataxin (FXN) polypeptide can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 4.

In some aspects, the invention provides a nucleic acid sequence comprising SEQ ID NO: 3 or a variant thereof for treating cardiomyopathy associated with Friedreich ataxia.

The variants include, for instance, naturally-occurring variants due to allelic variations between individuals (e.g., polymorphisms), alternative splicing forms, in particular transcript variants 2 and 3 (accession numbers NM_001161706 and NM_181425), etc. The term variant also includes FXN gene sequences from other sources or organisms. Variants are preferably substantially homologous to SEQ ID NO: 3, i.e., exhibit a nucleotide sequence identity of typically at least about 75%, preferably at least about 85%, more preferably at least about 90%, more preferably at least about 95%, 96%, 97%, 98%, or 99% with SEQ ID NO: 3. Variants of a FXN gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In some aspects, the FXN-encoding nucleic acid is a fragment of the SEQ ID NO: 3.

In some aspects, a sequence known as mitochondrion-targeting signal or mitochondrial targeting signal may be added to the FXN-encoding sequence or variant thereof, including, for example the FXN-encoding sequence “81-210”. Sequences known as mitochondrion-targeting signal or mitochondrial targeting signal are referred to as MTS by the skilled person.

A MTS sequence can be identified within a protein or nucleic acid sequence by a person of ordinary skill in the art.

Most mitochondrion-targeting peptides consist of a N-terminal pre-sequence of about 15 to 100 residues, preferably of about 20 to 80 residues. They are enriched in arginine, leucine, serine and alanine. Mitochondrial pre-sequences show a statistical bias of positively charged amino acid residues, provided mostly through arginine residues; very few sequences contain negatively charged amino acids. Mitochondrion-targeting peptides also share an ability to form an amphiphilic alpha-helix.

A complete description of a method to identify a MTS is available in: M. G. Claros, P. Vincens, 1996 (Eur. J. Biochem. 241, 779-786 (1996), “Computational method to predict mitochondrially imported proteins and their targeting sequences”), the content of which is herein incorporated by reference.

### Inverted Terminal Repeat Sequences

By “inverted terminal repeats” or “ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AA2, AAV3, AAV4, AAV5, AAV6, etc. Furthermore, 5′ and 3′ ITRs which flank a selected nucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh10 ITR sequence.

In some aspects, an rAAV vector of the present disclosure can comprise, consist essentially of, or consist of AAV2 ITR sequences. In some aspects, an rAAV vector of the present disclosure can comprise, consist essentially of, or consist of AAV2 ITR sequences or a modified AAV2 ITR sequence.

In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1, or complement thereof. In some aspects, a first ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2, or complement thereof.

In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1, or complement thereof. In some aspects, a second ITR can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2, or complement thereof.

### Promoter and Enhancer Sequences

The term “promoter” and “promoter sequence” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind.

The selected nucleotide sequence, such as a frataxin-encoding nucleotide sequence, is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, CAG, MCK (muscle creatine kinase), the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), chicken β-actin (CBA) promoter, rous sarcoma virus (RSV) promoter, synthetic promoters, hybrid promoters, and the like. The promoters can be of human origin or from other species, including from mice. In addition, sequences derived from nonviral genes, such as the murine metallothionein gene, will also find use herein. Such promoter sequences are commercially available from, e. g. Stratagene (San Diego, CA).

Examples of heterologous promoters include the CMV promoter.

Examples of inducible promoters include DNA responsive elements for ecdysone, tetracycline, hypoxia andaufin.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a CMV enhancer linked to a CBA promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a gene (i.e. a transgene) that is under the control of a promoter with which it is spatially connected. A promoter can be positioned 5′ (upstream) or 3′ (downstream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

In some aspects, an enhancer sequence can comprise, consist essentially of, or consist of a human cytomegalovirus (CMV) enhancer sequence. A CMV enhancer sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5.

In some aspects, a promoter sequence can comprise, consist essentially of, or consist of a chicken β-actin promoter sequence. A chicken β-actin promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 6.

### Intron Sequences

In some aspects, an intron sequence can comprise any intron sequence known in the art. In some aspects, the intron sequence can be a chimeric intron sequence. In some aspects, a chimeric intron sequence can comprise, consist essentially of a nucleic acid sequence at least 65%, 70%, 75%, 80%, 85%, 90%, 95%, 96%, 97%, 98%, 99% or 100% (or any percentage in between) identical to SEQ ID NO: 7.

### Polyadenylation Sequences

In some aspects, a polyadenylation (polyA) sequence can comprise any polyA sequence known in the art. In some aspects, a polyA sequence can comprise, consist essentially of, or consist of a β-globin polyA sequence. A β-globin polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 8.

### AAV Viral Vectors

AAV vectors of the disclosure can be packaged as an AAV viral vector.

An “AAV viral vector” refers to a viral particle composed of at least one AAV capsid protein and an encapsidated polynucleotide AAV vector. Thus, production of an AAV viral vector necessarily includes production of an AAV vector. The term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3.

AAV viral vectors useful in the practice of the present invention can be constructed utilizing methodologies well known in the art of molecular biology. Typically, AAV viral vectors carrying transgenes are assembled from polynucleotides encoding the transgene, suitable regulatory elements and elements necessary for production of viral proteins which mediate cell transduction.

The terms “gene transfer” or “gene delivery” refer to methods or systems for reliably inserting foreign DNA into host cells. Such methods can result in transient expression of non integrated transferred DNA, extrachromosomal replication and expression of transferred replicons (e.g. episomes), or integration of transferred genetic material into the genomic DNA of host cells.

Examples of viral vectors include but are not limited to adenoviral, retroviral, lentiviral, herpesvirus and adeno-associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In one embodiment, adeno-associated viral (AAV) vectors are employed.

In other embodiments, the AAV vector is AAV1, AAV2, AAV4, AAV5, AAV6, AAV7, AAV8, AAV9, AAV10, AAV11, AAV12, AAV13, AAVPHP.B, AAVrh74, AAVrh.10 or any other serotypes of AAV known in the art that can infect humans, monkeys or other species.

In an exemplary embodiment, the AAV vector is an AAVrh10 vector.

By an “AAV vector” is meant a vector derived from an adeno-associated virus serotype, including without limitation, AAV1, AAV2, AAV4, AAV5, AAV6, AAV7, AAV8, AAV9, AAV10, AAV11, AAV12, AAV13, AAVPHP.B, AAVrh74 or AAVrh.10. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the FXN gene) and a transcriptional termination region.

The control elements are selected to be functional in a mammalian cell. The resulting construct which contains the operatively linked components is bounded (5′ and 3′) with functional AAV ITR sequences.

Particularly preferred are vectors derived from AAV serotypes having tropism for and high transduction efficiencies in cells of the mammalian myocardium, particularly cardiomyocytes and cardiomyocyte progenitors. A review and comparison of transduction efficiencies of different serotypes is provided in Cearley C N et al., 2008. In other non-limiting examples, preferred vectors include vectors derived from any serotypes like AAV1, AAV2, AAV3, AAV4, AA5, AAV6, AAV7, AAV8, AAV9, or AAVrh10, which have also been shown to transduce cells of cardiomyocytes.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g. U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e. g. U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e. g., Edge, 1981; Nambair et al., 1984; Jay et al., 1984. In order to produce AAV virions, an AAV expression vector is introduced into a suitable host cell using known techniques, such as by transfection. A number of transfection techniques are generally known in the art. See, e. g., Graham et al., 1973; Sambrook et al. (1989) Molecular Cloning, a laboratory manual, Cold Spring Harbor Laboratories, New York, Davis et al. (1986) Basic Methods in Molecular Biology, Elsevier, and Chu et al., 1981. Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

For instance, a preferred viral vector, such as AAVrh10, comprises, in addition to a FXN encoding nucleic acid sequence, the backbone of AAV vector with ITR derived from AAV2, the promoter, such as the mouse PGK (phosphoglycerate kinase) gene or the cytomegalovirus/β-actin hybrid promoter (CAG) consisting of the enhancer from the cytomegalovirus immediate gene, the promoter, splice donor and intron from the chicken β-actin gene, the splice acceptor from rabbit β-globin, or any promoter such as PGK, CAG, MCK. AAV viral vectors of the disclosure comprise: i) an AAV vector described herein; and ii) an AAV capsid protein.

In some aspects, an AAV capsid protein can be any AAV capsid protein. In some aspects, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some aspects, the AAV capsid protein is an AAVrh.10 capsid protein.

### Methods of Treatment

A first object of the invention relates a method for treating or preventing cardiomyopathy in a subject in need thereof, comprising administering to said subject a therapeutically effective amount of an AAV vector which comprises a nucleic acid sequence encoding a frataxin (FXN) polypeptide or a fragment thereof.

In a particular embodiment, the cardiomyopathy may be a dilated cardiomyopathy, a hypertrophic cardiomyopathy, a restrictive cardiomyopathy or an ischemic cardiomyopathy.

In another particular embodiment, the cardiomyopathy may be a cardiomyopathy due to a deficiency of fatty oxidation, including but not limited to primary carnitine deficiency, LCHAD, translocase, VLCAD.

In another particular embodiment, the cardiomyopathy may be a cardiomyopathy associated with Friedreich ataxia.

In a particular embodiment, the gene encoded by a nucleic acid sequence in an AAV vector may be a nuclear gene encoding a subunit of pyruvate dehydrogenase complex, a nuclear or a mitochondrial gene coding for a subunit of Complex I, III, IV or V involved in the oxidative phosphorylation; a mitochondrial gene encoding transfer RNA, a gene involved in the biogenesis of mitochondria such as SIRTI, a gene involved in the fusion of mitochondria such as OPAI, a gene involved in the fission of mitochondria such as FISI or a gene involved in the oxidation of fatty acid such as the very long-chain specific acyl-CoA dehydrogenase.

In a particular embodiment, the gene encoded by a nucleic acid sequence in an AAV vector is the frataxin (FXN) gene.

As used herein in its broadest meaning, the term “preventing” or “prevention” refers to preventing the disease or condition from occurring in a subject which has not yet been diagnosed as having it or which does not have any clinical symptoms.

As used herein, the term “treating” or “treatment”, as used herein, means reversing, alleviating, or inhibiting the progress of the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a patient is such an amount which induces, ameliorates, stabilises, slows down the progression or otherwise causes an improvement in the pathological symptoms, disease progression or physiological conditions associated with or resistance to succumbing to a disorder.

As used herein, the term “subject” denotes a mammal, such as a rodent, a feline, a canine, and a primate. In some aspects, a subject according to the invention is a human. In the context of the present invention, a “subject in need thereof” denotes a subject, preferably a human, and more particularly a subject with a cardiomyopathy associated with Friedreich ataxia. Subject with a cardiomyopathy associated with Friedreich ataxia presents some cardiac symptoms which may be, but are not limited to, a decrease of ejection fraction, increase of ventricular mass or cardiac hypertrophy. Thus, the method of the invention will be very useful to treat a subject with such disease (Friedreich ataxia) presenting such symptoms.

As used herein, the term “gene” refers to a polynucleotide containing at least one open reading frame that is capable of encoding a particular polypeptide or protein after being transcribed and translated.

As used herein, the terms “coding sequence”, “a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

In a particular embodiment, the invention relates to a method for preventing or treating a cardiomyopathy associated with Friedreich ataxia in a subject in need thereof, comprising administering to said subject a therapeutically effective amount of an AAV vector which comprises a frataxin (FXN) encoding nucleic acid.

In a particular embodiment, the invention relates to a method for treating a cardiomyopathy associated with Friedreich ataxia in a subject in need thereof, comprising administering to said subject a therapeutically effective amount of an AAV vector which comprises a frataxin (FXN) encoding nucleic acid.

In a particular embodiment, the invention relates to a method for reversing or stabilizing symptoms of cardiomyopathy associated with Friedreich ataxia in a subject in need thereof, comprising administering to said subject a therapeutically effective amount of an AAV vector which comprises a frataxin (FXN) encoding nucleic acid.

As used herein, the term “reversing symptoms of cardiomyopathy associated with Friedreich ataxia” denotes the restoration of cardiac function by, for example, improving ejection fraction and/or decreasing the ventricular mass in a subject in need thereof.

In a particular embodiment, the invention relates to a method for reversing the dysfunction of cardiac mitochondria associated with Friedreich ataxia in a subject in need thereof, comprising administering to said subject a therapeutically effective amount of an AAV vector which comprises a frataxin (FXN) encoding nucleic acid.

In a particular embodiment, the invention relates to a method for improving the cardiac mitochondria associated with Friedreich ataxia in a subject in need thereof, comprising administering to said subject a therapeutically effective amount of an AAV vector which comprises a frataxin (FXN) encoding nucleic acid.

In a particular embodiment, the invention relates to a method for restoring cardiac function in a subject suffering of a cardiomyopathy associated with Friedreich ataxia comprising administering to said subject of a therapeutically effective amount of an AAV vector which comprises a frataxin (FXN) encoding nucleic acid.

In a particular embodiment, the invention relates to a method for improving cardiac function in a subject suffering of a cardiomyopathy associated with Friedreich ataxia comprising administering to said subject a therapeutically effective amount of an AAV vector which comprises a frataxin (FXN) encoding nucleic acid.

In a particular embodiment, the invention relates to a method for treating a cardiomyopathy associated with Friedreich ataxia in an asymptomatic or pre-symptomatic subject in need thereof, comprising administering to said subject a therapeutically effective amount of an AAV vector which comprises a frataxin (FXN) encoding nucleic acid.

In another particular embodiment, the invention relates to a method for treating a cardiomyopathy associated with Friedreich ataxia in a symptomatic subject in need thereof, comprising administering to said subject a therapeutically effective amount of an AAV vector which comprises a frataxin (FXN) encoding nucleic acid.

As used herein, the terms “asymptomatic” or “pre-symptomatic” denotes a subject with the disease (Friedreich ataxia) as defined by a genetic diagnosis (see for review Lynch D R et al., Arch Neurol. 2002; 59:743-747) but with no detectable clinical cardiac symptom.

As used herein, the terms symptomatic denotes a subject with the disease (Friedreich ataxia) as defined by a genetic diagnosis and with the presence of cardiac symptoms (cardiac hypertrophy, fibrosis, decreased myocardiac perfusion reserve index, impaired cardiac or skeletal muscle mitochondrial respiratory chain function, subclinical cardiomyopathy, supraventricular arrhythmias, heart failure, systolic left ventricular dysfunction, fatigue . . . ).

The FXN gene encodes the protein frataxin. Frataxin is a protein localized to the mitochondrion. Frataxin is involved in assembly of iron-sulfur clusters by regulating iron entry and the activity of the cysteine desulfurase.

In some aspects, the invention relates to a method for use in the prevention or treatment of diseases associated with frataxin deficiency in a subject in need therefore, comprising to said subject administering a therapeutically effective amount of an AAV vector which comprises a nucleic acid encoding frataxin.

In some aspects, the invention relates a method for use in the prevention or treatment of cardiomyopathy in a subject in need thereof, comprising administering to said subject a therapeutically effective amount of an AAV vector which comprises a frataxin (FXN) encoding nucleic acid.

In some aspects, the subject experiences an increase in peak oxygen consumption (peak VO2) following AAV vector administration relative to a pre-AAV vector administration baseline. In some aspects, the peak VO2 in the subject is measured about 12 weeks, about 24 weeks, about 36 weeks, about 52 weeks, about 18 months, about 2 years, about 3 years, about 4 years, and/or about 5 years post-AAV vector administration. In some aspects, VO2 is a measure depicting the rate of oxygen consumption. In some aspects, VO2 is the volume of oxygen consumed per a unit of time.

In some aspects, peak VO2 is measured by cardiopulmonary exercise testing (CPET) using arm ergometry.

In some aspects, the subject experiences a decrease in Left Ventricular Mass index (LVMi) following AAV vector administration relative to a pre-AAV vector administration baseline.

In some aspects, LVMi is measured about 12 weeks, about 24 weeks, about 36 weeks, about 52 weeks post-AAV vector administration, about 18 months, about 2 years, about 3 years, about 4 years, and/or about 5 years post-AAV vector administration. In some aspects, LVMi is measured by cardiac MRI.

In some aspects, the subject, following AAV vector administration, experiences one or more of decreased global longitudinal strain, increased stroke volume, increased left ventricular ejection fraction (LVEF), and decreased or stable cardiac fibrosis as measured by cardiac MRI relative to a pre-AAV vector administration baseline.

In some aspects, the measurement by cardiac MRI occurs about 12 weeks, about 24 weeks, about 36 weeks, about 52 weeks, about 18 months, about 2 years, about 3 years, about 4 years, and/or about 5 years post-AAV vector administration.

In some aspects, the subject, following AAV vector administration, experiences decreased serum NTproBNP, hsTNT, and/or CK-MB levels relative to a pre-AAV vector administration baseline.

In some aspects, the subject, following AAV vector administration, experiences decreased cardiac arrythmias relative to a pre-AAV vector administration baseline. In some aspects, the subject, following AAV vector administration, experiences fewer incidences of cardiac arrythmias relative to a pre-AAV vector administration baseline.

In some aspects, cardiac arrythmias are evaluated by remote cardiac rhythm monitoring.

In some aspects, the subject, following AAV vector administration, experiences decreased fatigue as measured by the Modified Fatigue Impact Scale (MFIS) or Fatigue Severity Scale (FSS) relative to a pre-AAV vector administration baseline.

In some aspects, the subject, following AAV vector administration, experiences improvements in exertional symptoms (during CPET via arm ergometry) as measured by the Modified Borg Dyspnea, Borg Rating of Perceived Exhaustion (RPE), and the angina scale using CPET relative to a pre-AAV vector administration baseline.

It is known that Friedreich Ataxia heterozygotes, those individuals with one normal FXN allele, have no clinical manifestations of FA. Whereas FA homozygotes, individuals having two abnormal and/or mutated FXN alleles, experience symptoms. Analysis of the average FXN levels in autopsy samples of the hearts of 5 healthy individuals demonstrated normal levels of FXN in the human heart are 59±5 ng/mg protein. The average cardiac frataxin protein levels in FA homozygotes is estimated to be about 9.4 ng/mg. Further, it is known that FA heterozygotes have 30-80% of normal levels (19-50 ng/mg frataxin protein) of FXN relative to healthy individuals. Without wishing to be bound by theory, it is reasonable to expect that increasing the level of frataxin in an FA homozygote to a level near or above the level of an FA heterozygote or a healthy individual will result in improvement of symptoms experiences by FA homozygotes. In some aspects, following AAV vector administration of FXN-encoding vectors disclosed herein, the patient experiences an increase in frataxin protein expression. In some aspects, the increased frataxin expression occurs in the heart.

In some aspects, the amount of frataxin in the heart of the subject following AAV vector administration increases by about 1 ng/mg, about 2 ng/mg, about 3 ng/mg, about 4 ng/mg, about 5 ng/mg, about 6 ng/mg, about 7 ng/mg, about 8 ng/mg, about 9 ng/mg, about ng/mg, about 11 ng/mg, about 12 ng/mg, about 13 ng/mg, about 14 ng/mg, about 15 ng/mg, about 20 ng/mg, about 25 ng/mg, about 30 ng/mg, about 35 ng/mg, about 40 ng/mg, about 45 ng/mg, about 50 ng/mg, about 60 ng/mg, about 70 ng/mg, about 80 ng/mg, about 90 ng/mg, about 100 ng/mg, about 200 ng/mg, about 300 ng/mg, about 400 ng/mg, or about 500 ng/mg relative to a pre-administration baseline.

In some aspects, the subject, following AAV vector administration, experiences increased FXN expression levels relative to a pre-AAV vector administration baseline. In some aspects, increased FXN levels refers to increased mRNA levels of FXN. In some aspects, increased FXN levels refer to increased frataxin (FXN) protein levels.

In some aspects, the FXN levels are myocardial FXN expression levels. In some aspects, myocardial FXN expression is measured by cardiac biopsy.

In some aspects, the subject experiences an at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 150%, at least about 200%, at least about 300%, at least about 400%, or at least 500% increase in FXN expression levels relative to a pre-AAV vector administration baseline.

In some aspects, the subject, following AAV vector administration, experiences improvements or stabilizations in the Friedreich's Ataxia Rating Scale (FARS) or modified Friedreich's Ataxia Rating Scale (mFARS) relative to a pre-AAV vector administration baseline.

In some aspects, the subject, following AAV vector administration, experiences improvements or stabilizations in the Scale for Assessment and Rating of Ataxia (SARA) relative to a pre-AAV vector administration baseline.

In some aspects, the subject, following AAV vector administration, experiences improvements or stabilizations in the Shortness of Breath-Daily Activities Score (SOBDA) relative to a pre-AAV vector administration baseline.

In some aspects, the subject, following AAV vector administration, experiences improvements or stabilizations in the Seattle Angina Questionnaire (SAQ) relative to a pre-AAV vector administration baseline.

### Delivery of the Vectors

It is herein provided a method for treating cardiomyopathy associated with Friedreich ataxia in a subject, said method comprising: (a) providing an AAV vector as defined above, which comprises a nucleic acid sequence encoding a frataxin (FXN) polypeptide or a fragment thereof; and (b) delivering the AAV vector to the subject in need thereof and whereby FXN is expressed by the transduced cells at a therapeutically effective level.

The preferred doses and regimen may be determined by a physician, and depend on the age, sex, weight, of the subject, and the stage of the disease.

In some aspects, the AAV vector is administered to the subject intravenously (IV). In some aspects, the IV infusion occurs over at least about 5 minutes, at least about 10 minutes, at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes. In some aspects, the IV infusion occurs over 60 minutes.

In some aspects, the subject is administered an AAV vector of the disclosure at a therapeutically effective dosage. In some aspects, the dosage is between about 1.0×10¹⁰ genome copies (gc) per kilogram (kg) (gc/kg) to about 9.9×10¹⁴ gc/kg. In some aspects, the dosage is between about 1.0×10¹⁰ gc/kg to about 6.0×10¹⁴ gc/kg. In some aspects, the dosage is between about 1.0×10¹⁰ gc/kg to about 6.0×10¹³ gc/kg. In some aspects, the dosage is between about 1.0×10¹⁰ gc/kg to about 9.9×10¹³ gc/kg. In some aspects, the dosage is between about 1.0×10¹⁰ gc/kg to about 9.9×10¹² gc/kg. In some aspects, the dosage is between about 1.0×10¹⁰ gc/kg to about 9.9×10¹¹ gc/kg. In some aspects, the dosage is between about 1.0×10¹¹ gc/kg to about 9.9×10¹⁴ gc/kg. In some aspects, the dosage is between about 1.0×10¹² gc/kg to about 9.9×10¹⁴ gc/kg. In some aspects, the dosage is between about 1.0×10¹³ gc/kg to about 9.9×10¹⁴ gc/kg. In some aspects, the dosage is between about 1.0×10¹⁴ gc/kg to about 9.9×10¹⁴ gc/kg.

In some aspects, the dosage is about 1.0×10¹⁰ gc/kg, about 1.1×10¹⁰ gc/kg, about 1.2×10¹⁰ gc/kg, about 1.3×10¹⁰ gc/kg, about 1.4×10¹⁰ gc/kg, about 1.5×10¹⁰ gc/kg, about 1.6×10¹⁰ gc/kg, about 1.7×10¹⁰ gc/kg, about 1.8×10¹⁰ gc/kg, about 1.9×10¹⁰ gc/kg, about 2.0×10¹⁰ gc/kg, about 2.1×10¹⁰ gc/kg, about 2.2×10¹⁰ gc/kg, about 2.3×10¹⁰ gc/kg, about 2.4×10¹⁰ gc/kg, about 2.5×10¹⁰ gc/kg, about 2.6×10¹⁰ gc/kg, about 2.7×10¹⁰ gc/kg, about 2.8×10¹⁰ gc/kg, about 2.9×10¹⁰ gc/kg, about 3.0×10¹⁰ gc/kg, about 3.1×10¹⁰ gc/kg, about 3.2×10¹⁰ gc/kg, about 3.3×10¹⁰ gc/kg, about 3.4×10¹⁰ gc/kg, about 3.5×10¹⁰ gc/kg, about 3.6×10¹⁰ gc/kg, about 3.7×10¹⁰ gc/kg, about 3.8×10¹⁰ gc/kg, about 3.9×10¹⁰ gc/kg, about 4.0×10¹⁰ gc/kg, about 4.1×10¹⁰ gc/kg, about 4.2×10¹⁰ gc/kg, about 4.3×10¹⁰ gc/kg, about 4.4×10¹⁰ gc/kg, about 4.5×10¹⁰ gc/kg, about 4.6×10¹⁰ gc/kg, about 4.7×10¹⁰ gc/kg, about 4.8×10¹⁰ gc/kg, about 4.9×10¹⁰ gc/kg, about 5.0×10¹⁰ gc/kg, about 5.1×10¹⁰ gc/kg, about 5.2×10¹⁰ gc/kg, about 5.3×10¹⁰ gc/kg, about 5.4×10¹⁰ gc/kg, about 5.5×10¹⁰ gc/kg, about 5.6×10¹⁰ gc/kg, about 5.7×10¹⁰ gc/kg, about 5.8×10¹⁰ gc/kg, about 5.9×10¹⁰ gc/kg, about 6.0×10¹⁰ gc/kg, about 6.1×10¹⁰ gc/kg, about 6.2×10¹⁰ gc/kg, about 6.3×10¹⁰ gc/kg, about 6.4×10¹⁰ gc/kg, about 6.5×10¹⁰ gc/kg, about 6.6×10¹⁰ gc/kg, about 6.7×10¹⁰ gc/kg, about 6.8×10¹⁰ gc/kg, about 6.9×10¹⁰ gc/kg, about 7.0×10¹⁰ gc/kg, about 7.1×10¹⁰ gc/kg, about 7.2×10¹⁰ gc/kg, about 7.3×10¹⁰ gc/kg, about 7.4×10¹⁰ gc/kg, about 7.5×10¹⁰ gc/kg, about 7.6×10¹⁰ gc/kg, about 7.7×10¹⁰ gc/kg, about 7.8×10¹⁰ gc/kg, about 7.9×10¹⁰ gc/kg, about 8.0×10¹⁰ gc/kg, about 8.1×10¹⁰ gc/kg, about 8.2×10¹⁰ gc/kg, about 8.3×10¹⁰ gc/kg, about 8.4×10¹⁰ gc/kg, about 8.5×10¹⁰ gc/kg, about 8.6×10¹⁰ gc/kg, about 8.7×10¹⁰ gc/kg, about 8.8×10¹⁰ gc/kg, about 8.9×10¹⁰ gc/kg, about 9.0×10¹⁰ gc/kg, about 9.1×10¹⁰ gc/kg, about 9.2×10¹⁰ gc/kg, about 9.3×10¹⁰ gc/kg, about 9.4×10¹⁰ gc/kg, about 9.5×10¹⁰ gc/kg, about 9.6×10¹⁰ gc/kg, about 9.7×10¹⁰ gc/kg, about 9.8×10¹⁰ gc/kg, or about 9.9×10¹⁰ gc/kg.

In some aspects, the dosage is about 1.0×10¹¹ gc/kg, about 1.1×10¹¹ gc/kg, about 1.2×10¹¹ gc/kg, about 1.3×10¹¹ gc/kg, about 1.4×10¹¹ gc/kg, about 1.5×10¹¹ gc/kg, about 1.6×10¹¹ gc/kg, about 1.7×10¹¹ gc/kg, about 1.8×10¹¹ gc/kg, about 1.9×10¹¹ gc/kg, about 2.0×10¹¹ gc/kg, about 2.1×10¹¹ gc/kg, about 2.2×10¹¹ gc/kg, about 2.3×10¹¹ gc/kg, about 2.4×10¹¹ gc/kg, about 2.5×10¹¹ gc/kg, about 2.6×10¹¹ gc/kg, about 2.7×10¹¹ gc/kg, about 2.8×10¹¹ gc/kg, about 2.9×10¹¹ gc/kg, about 3.0×10¹¹ gc/kg, about 3.1×10¹¹ gc/kg, about 3.2×10¹¹ gc/kg, about 3.3×10¹¹ gc/kg, about 3.4×10¹¹ gc/kg, about 3.5×10¹¹ gc/kg, about 3.6×10¹¹ gc/kg, about 3.7×10¹¹ gc/kg, about 3.8×10¹¹ gc/kg, about 3.9×10¹¹ gc/kg, about 4.0×10¹¹ gc/kg, about 4.1×10¹¹ gc/kg, about 4.2×10¹¹ gc/kg, about 4.3×10¹¹ gc/kg, about 4.4×10¹¹ gc/kg, about 4.5×10¹¹ gc/kg, about 4.6×10¹¹ gc/kg, about 4.7×10¹¹ gc/kg, about 4.8×10¹¹ gc/kg, about 4.9×10¹¹ gc/kg, about 5.0×10¹¹ gc/kg, about 5.1×10¹¹ gc/kg, about 5.2×10¹¹ gc/kg, about 5.3×10¹¹ gc/kg, about 5.4×10¹¹ gc/kg, about 5.5×10¹¹ gc/kg, about 5.6×10¹¹ gc/kg, about 5.7×10¹¹ gc/kg, about 5.8×10¹¹ gc/kg, about 5.9×10¹¹ gc/kg, about 6.0×10¹¹ gc/kg, about 6.1×10¹¹ gc/kg, about 6.2×10¹¹ gc/kg, about 6.3×10¹¹ gc/kg, about 6.4×10¹¹ gc/kg, about 6.5×10¹¹ gc/kg, about 6.6×10¹¹ gc/kg, about 6.7×10¹¹ gc/kg, about 6.8×10¹¹ gc/kg, about 6.9×10¹¹ gc/kg, about 7.0×10¹¹ gc/kg, about 7.1×10¹¹ gc/kg, about 7.2×10¹¹ gc/kg, about 7.3×10¹¹ gc/kg, about 7.4×10¹¹ gc/kg, about 7.5×10¹¹ gc/kg, about 7.6×10¹¹ gc/kg, about 7.7×10¹¹ gc/kg, about 7.8×10¹¹ gc/kg, about 7.9×10¹¹ gc/kg, about 8.0×10¹¹ gc/kg, about 8.1×10¹¹ gc/kg, about 8.2×10¹¹ gc/kg, about 8.3×10¹¹ gc/kg, about 8.4×10¹¹ gc/kg, about 8.5×10¹¹ gc/kg, about 8.6×10¹¹ gc/kg, about 8.7×10¹¹ gc/kg, about 8.8×10¹¹ gc/kg, about 8.9×10¹¹ gc/kg, about 9.0×10¹¹ gc/kg, about 9.1×10¹¹ gc/kg, about 9.2×10¹¹ gc/kg, about 9.3×10¹¹ gc/kg, about 9.4×10¹¹ gc/kg, about 9.5×10¹¹ gc/kg, about 9.6×10¹¹ gc/kg, about 9.7×10¹¹ gc/kg, about 9.8×10¹¹ gc/kg, or about 9.9×10¹¹ gc/kg.

In some aspects, the dosage is about 1.0×10¹² gc/kg, about 1.1×10¹² gc/kg, about 1.2×10¹² gc/kg, about 1.3×10¹² gc/kg, about 1.4×10¹² gc/kg, about 1.5×10¹² gc/kg, about 1.6×10¹² gc/kg, about 1.7×10¹² gc/kg, about 1.8×10¹² gc/kg, about 1.9×10¹² gc/kg, about 2.0×10¹² gc/kg, about 2.1×10¹² gc/kg, about 2.2×10¹² gc/kg, about 2.3×10¹² gc/kg, about 2.4×10¹² gc/kg, about 2.5×10¹² gc/kg, about 2.6×10¹² gc/kg, about 2.7×10¹² gc/kg, about 2.8×10¹² gc/kg, about 2.9×10¹² gc/kg, about 3.0×10¹² gc/kg, about 3.1×10¹² gc/kg, about 3.2×10¹² gc/kg, about 3.3×10¹² gc/kg, about 3.4×10¹² gc/kg, about 3.5×10¹² gc/kg, about 3.6×10¹² gc/kg, about 3.7×10¹² gc/kg, about 3.8×10¹² gc/kg, about 3.9×10¹² gc/kg, about 4.0×10¹² gc/kg, about 4.1×10¹² gc/kg, about 4.2×10¹² gc/kg, about 4.3×10¹² gc/kg, about 4.4×10¹² gc/kg, about 4.5×10¹² gc/kg, about 4.6×10¹² gc/kg, about 4.7×10¹² gc/kg, about 4.8×10¹² gc/kg, about 4.9×10¹² gc/kg, about 5.0×10¹² gc/kg, about 5.1×10¹² gc/kg, about 5.2×10¹² gc/kg, about 5.3×10¹² gc/kg, about 5.4×10¹² gc/kg, about 5.5×10¹² gc/kg, about 5.6×10¹² gc/kg, about 5.7×10¹² gc/kg, about 5.8×10¹² gc/kg, about 5.9×10¹² gc/kg, about 6.0×10¹² gc/kg, about 6.1×10¹² gc/kg, about 6.2×10¹² gc/kg, about 6.3×10¹² gc/kg, about 6.4×10¹² gc/kg, about 6.5×10¹² gc/kg, about 6.6×10¹² gc/kg, about 6.7×10¹² gc/kg, about 6.8×10¹² gc/kg, about 6.9×10¹² gc/kg, about 7.0×10¹² gc/kg, about 7.1×10¹² gc/kg, about 7.2×10¹² gc/kg, about 7.3×10¹² gc/kg, about 7.4×10¹² gc/kg, about 7.5×10¹² gc/kg, about 7.6×10¹² gc/kg, about 7.7×10¹² gc/kg, about 7.8×10¹² gc/kg, about 7.9×10¹² gc/kg, about 8.0×10¹² gc/kg, about 8.1×10¹² gc/kg, about 8.2×10¹² gc/kg, about 8.3×10¹² gc/kg, about 8.4×10¹² gc/kg, about 8.5×10¹² gc/kg, about 8.6×10¹² gc/kg, about 8.7×10¹² gc/kg, about 8.8×10¹² gc/kg, about 8.9×10¹² gc/kg, about 9.0×10¹² gc/kg, about 9.1×10¹² gc/kg, about 9.2×10¹² gc/kg, about 9.3×10¹² gc/kg, about 9.4×10¹² gc/kg, about 9.5×10¹² gc/kg, about 9.6×10¹² gc/kg, about 9.7×10¹² gc/kg, about 9.8×10¹² gc/kg, or about 9.9×10¹² gc/kg.

In some aspects, the dosage is about 1.0×10¹¹ gc/kg, about 1.1×10¹¹ gc/kg, about 1.2×10¹¹ gc/kg, about 1.3×10¹¹ gc/kg, about 1.4×10¹¹ gc/kg, about 1.5×10¹¹ gc/kg, about 1.6×10¹¹ gc/kg, about 1.7×10¹¹ gc/kg, about 1.8×10¹¹ gc/kg, about 1.9×10¹¹ gc/kg, about 2.0×10¹¹ gc/kg, about 2.1×10¹¹ gc/kg, about 2.2×10¹¹ gc/kg, about 2.3×10¹¹ gc/kg, about 2.4×10¹¹ gc/kg, about 2.5×10¹¹ gc/kg, about 2.6×10¹¹ gc/kg, about 2.7×10¹¹ gc/kg, or about 2.8×10¹¹ gc/kg.

In some aspects, the dosage is about 5.0×10¹¹ gc/kg, about 5.1×10¹¹ gc/kg, about 5.2×10¹¹ gc/kg, about 5.3×10¹¹ gc/kg, about 5.4×10¹¹ gc/kg, about 5.5×10¹¹ gc/kg, about 5.6×10¹¹ gc/kg, about 5.7×10¹¹ gc/kg, about 5.8×10¹¹ gc/kg, about 5.9×10¹¹ gc/kg, about 6.0×10¹¹ gc/kg, about 6.1×10¹¹ gc/kg, about 6.2×10¹¹ gc/kg, about 6.3×10¹¹ gc/kg, about 6.4×10¹¹ gc/kg, about 6.5×10¹¹ gc/kg, or about 6.6×10¹¹ gc/kg.

In some aspects, the dosage is about 1.0×10¹² gc/kg, about 1.1×10¹² gc/kg, about 1.2×10¹² gc/kg, about 1.3×10¹² gc/kg, about 1.4×10¹² gc/kg, about 1.5×10¹² gc/kg, about 1.6×10¹² gc/kg, about 1.7×10¹² gc/kg, about 1.8×10¹² gc/kg, about 1.9×10¹² gc/kg, or about 2.0×10¹² gc/kg.

In some aspects, the dosage is about 1.8×10¹¹ gc/kg. In some aspects, the dosage is about 5.6×10¹¹ gc/kg. In some aspects, the dosage is about 5.7×10¹¹ gc/kg. In some aspects, the dosage is about 1.8×10¹² gc/kg.

In some aspects, the dosage is measured by quantitative polymerase chain reaction (qPCR) titer. In some aspects, the dosage is measured by droplet digital polymerase chain reaction (ddPCR) titer.

In some aspects, the therapeutically effective dosage can be tailored for each AAV capsid serotype. In some aspects, the therapeutically effective dosage is tailored to account for differences in cardiac tropism for distinct AAV capsid serotypes.

In some aspects, the subject is administered a single dose of AAV vector. In some aspects, the subject is further administered a second, third, fourth, or fifth dosage of the AAV vector. In some aspects, second and subsequent administrations of AAV vector can be at a different dosage from the first dosage.

In some aspects, the subject is further administered prednisone along with AAV vector administration.

In some aspects, the prednisone is administered at a dosage of:
40 mg, once daily 24 hours prior to AAV viral vector administration;
40 mg once daily for week 1 through week 8 post-AAV viral vector administration;
30 mg once daily for week 9 post-AAV viral vector administration;
20 mg once daily for week 10 post-AAV viral vector administration;
10 mg once daily for week 11 post-AAV viral vector administration;
5 mg once daily for week 12 post-AAV viral vector administration;
2.5 mg once daily for week 13 post-AAV viral vector administration; and
2.5 mg every other day for week 14 post-AAV viral vector administration.

In some aspects, the AAV vector is delivered directly into the myocardium by epicardiac injection followed by minithoracotomy, by intracoronary injection, by endomyocardic injection, by subepicardial or epicardial injection or other type of injection useful in the heart.

Additional routes of administration may also comprise local application of the vector under direct visualization, e.g., superficial cortical application, or other nonstereotactic application. The vector may be delivered intrathecally, in the ventricles or by intravenous injection.

The target cells of the vectors of the present invention are cells of the myocardium of a subject afflicted with a cardiomyopathy associated with Friedreich ataxia. Preferably the subject is a human being, adult or child.

However the invention also encompasses delivering the vector to biological models of the disease. In that case, the biological model may be any mammal at any stage of development at the time of delivery, e.g., embryonic, fetal, infantile, juvenile or adult. Furthermore, the target myocardium cells may be essentially from any source, especially any cells derived from hiPS from FRDA patients, nonhuman primates and mammals of the orders Rodenta (mice, rats, rabbit, hamsters), Carnivora (cats, dogs), and Arteriodactyla (cows, pigs, sheep, goats, horses) as well as any other non-human system (e. g. zebrafish model system).

The vectors used herein may be formulated in any suitable vehicle for delivery. For instance, they may be placed into a pharmaceutically acceptable suspension, solution or emulsion. Suitable media include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

In some aspects, the invention relates to a vector which comprises a FXN encoding nucleic acid for use in treatment or prevention of cardiomyopathy associated with Friedreich ataxia in a subject wherein the AAV vector is delivering the subject in need thereof and wherein FXN is expressed by the transduced cells at a therapeutically effective level.

In a particular embodiment, the invention relates to a vector which comprises a FXN encoding nucleic acid for reversing symptoms of cardiomyopathy associated with Friedreich ataxia in a subject in need thereof wherein the AAV vector is delivering the subject in need thereof and wherein FXN is expressed by the transduced cells at a therapeutically effective level.

### Non Viral Vectors

In a particular embodiment, the vector use according to the invention is a non viral vector. Typically, the non viral vector may be a plasmid which includes nucleic acid sequences encoding the FXN gene, or variants thereof, as described above.

### Pharmaceutical Compositions

In some aspects, the invention concerns a pharmaceutical composition for preventing or treating cardiomyopathy associated with Friedreich ataxia in a subject in need thereof, which comprises a therapeutically effective amount of an AAV vector which comprises a FXN encoding nucleic acid.

By a “therapeutically effective amount” is meant a sufficient amount of the AAV vector of the invention to treat a cardiomyopathy associated with Friedreich ataxia at a reasonable benefit/risk ratio applicable to any medical treatment.

It will be understood that the single dosage or the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient;

the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the AAV vector according to the invention may be in any form that is suitable for the selected mode of administration, for example, for intraventricular, intramyocardium, intracoronary or intravenous administration.

In the pharmaceutical compositions of the present invention for intramuscular, intravenous, intramyocardium, intracoronary or intraventricular administration, the active principle, alone or in combination with another active principle, can be administered in a unit administration form, as a mixture with conventional pharmaceutical supports, to animals and human beings.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The AAV vector according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Multiple doses can also be administered.

In some aspects, the invention relates to a pharmaceutical composition for treating or preventing diseases associated with frataxin deficiency in a subject in need therefore, comprising to said subject administering a therapeutically effective amount of a vector which comprises a nucleic acid encoding frataxin.

### EXAMPLES

The invention will be further illustrated by the following figures and examples. However, these examples and figures should not be interpreted in any way as limiting the scope of the present invention.

### Example 1: Pre-Clinical Assessment of Frataxin AAV Vector Therapy

For in vivo adeno-associated virus (AAV) gene therapy to be successful, it is critical to identify a minimal effective clinical dose, setting the therapeutic goal for a human clinical trial. Friedreich's ataxia (FA), a life-threatening disorder for which there is no disease-modifying therapy, is characterized by neurologic and cardiac dysfunction. Most FA patients are homozygous for a GAA trinucleotide expansion within the first intron of the frataxin (FXN) gene, which causes transcriptional silencing, leading to a reduction in the levels of FXN mRNA and protein. While progressive neurologic disease limits mobility, cardiomyopathy is the cause of death in nearly two-thirds of individuals. From prior studies in experimental animals, it is known that an adeno-associated virus serotype rh.10 expressing human FXN (AAVrh.10hFXN) effectively treats the cardiac manifestations of the disease in murine models of FA, but the therapeutic window is narrow, with high doses of the hFXN transgene associated with toxicity. As such, it is critical to establish the dose that will effectively treat cardiac manifestations of FA in subjects.

It is known that FA heterozygotes with one normal FXN allele have no clinical manifestations of FA. As such the level of FXN necessary to convert the heart of an FA homozygote to that of a heterozygote was estimated. Analysis of the average FXN levels in autopsy samples of the hearts of 5 individuals demonstrated normal levels of FXN in the human heart are 59±5 ng/mg protein. The average cardiac frataxin protein levels in FA homozygotes is estimated to be about 9.4 ng/mg. Further, it is known that FA heterozygotes have 30-80% of normal levels (19-50 ng/mg frataxin protein) of FXN. It was estimated that, to be effective, an AAV vector needs to provide >9 ng/mg frataxin protein expression. for effective therapy (heterozygote 19-50 ng/mg-FA homozygotes ˜10 ng/mg=9-40 ng/mg).

### Mouse Dosage Study

MCK (creatine kinase−/−fxn deficient) mice are a model of severe cardiac disease that completely lacks murine FXN protein in cardiac and skeletal muscle. MCK mice have progressive disease deterioration with death in untreated animals by wk 11.

To determine a dose of AAVrh.10hFXN necessary to produce >8.3 ng/mg in the heart, doses of 1.8×10¹¹, 5.7×10¹¹ or 1.8×10¹² gc/kg (qPCR determined) AAVrh.10hFXN were administered intravenously to 7-wk old MCK mice (n=20/dose; 10 females, 10 males). These doses were chosen because they are significantly lower than those previously shown to be toxic (14- to 140-fold). Following administration, hFXN protein levels in cardiac tissue, body weight, echocardiograms, cardiac function and mortality were assessed.

Expression of hFXN in Mouse Cardiac Tissue

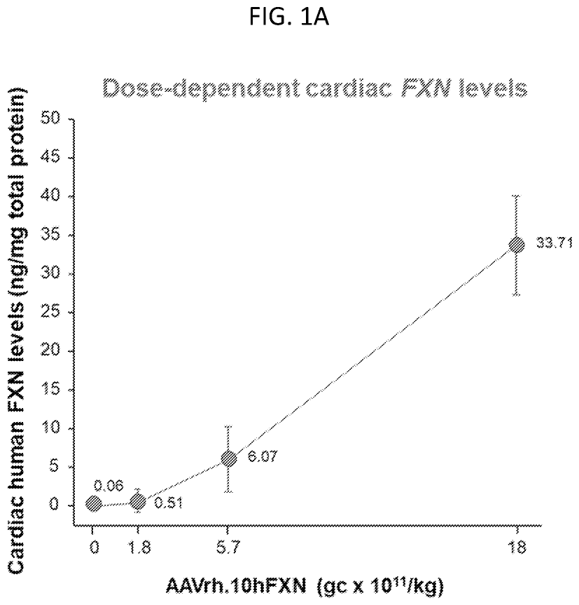

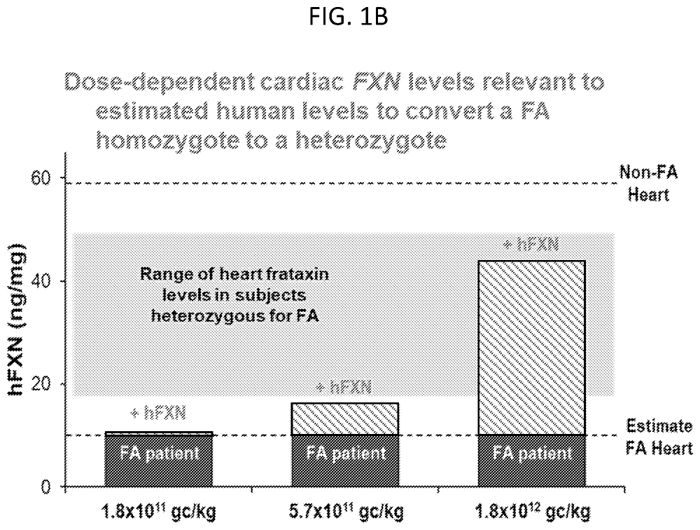

Assessment of hFXN total protein in cardiac tissue of MCK mice following administration of AAVrh.10hFXN showed that there was a dose-dependent increase in cardiac hFXN expression associated with increasing doses of intravenous AAVrh.10hFXN. As shown in FIG. 1A, intravenous administration of a 1.8×10¹¹ gc/kg, 5.7×10¹¹ gc/kg, and 18×10¹² gc/kg dose of AAVrh.10hFXN vector associated with cardiac hFXN protein levels of 0.51 ng/mg, 6.07 ng/mg, and 33.71 ng/mg, respectively. To establish if the cardiac levels observed in MCK mice are in the “therapeutic” range, these cardiac hFXN levels were added to the anticipated endogenous FXN levels in FA patients cited above. As shown in FIG. 1B, at 5.7×10¹¹ and 1.8×10¹² gc/kg, the increase in FXN levels when added to endogenous reached 26% and 73% of normal human heart FXN levels, respectively, and within the heterozygous range at the 1.8×10¹² gc/kg dose.

### Efficacy of Intravenous Administration on Body Weight of Mice

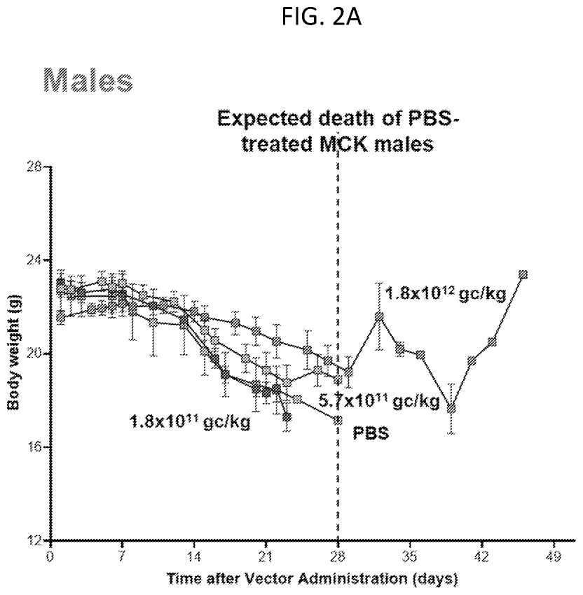

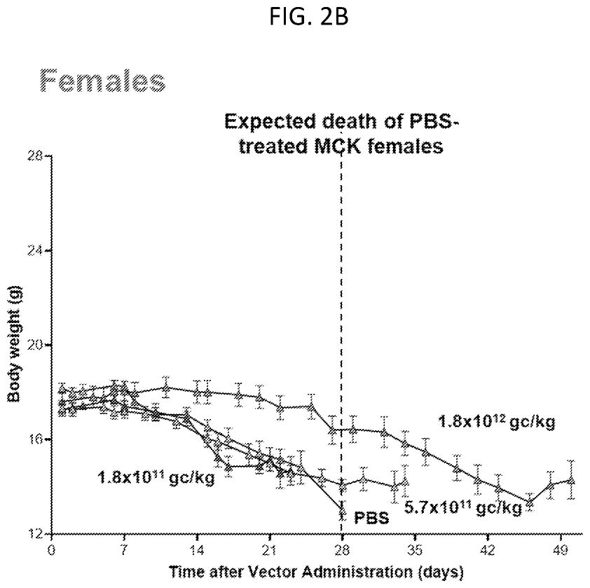

The body weight of MCK mice was monitored following intravenous administration of three doses of AAVrh.10hFXN (1.8×10¹¹ gc/kg, 5.7×10¹¹ gc/kg, and 1.8×10¹² gc/kg) and compared to mice administered PBS as a control. FIG. 2A shows the body weight of male mice administered three doses of vector compared to PBS. FIG. 2B shows the body weight of female mice administered three doses of vector compared to PBS control. Significant difference (p<0.001) between the PBS-treated and the 1.8×10¹² gc/kg AAVrh.10FXN cohorts (combined male and female; repeated measures ANOVA with effect of time) was observed.

### Enhanced Dosage Study

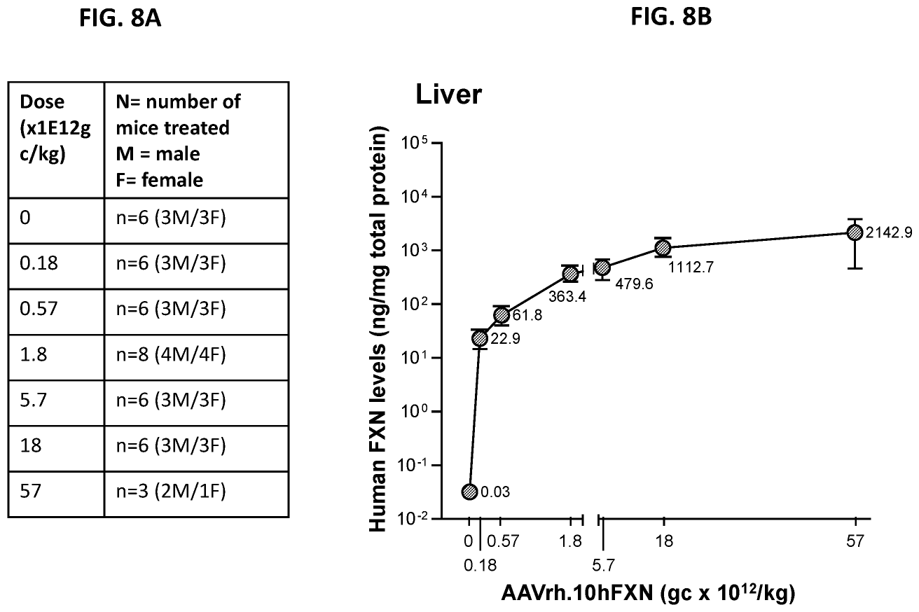

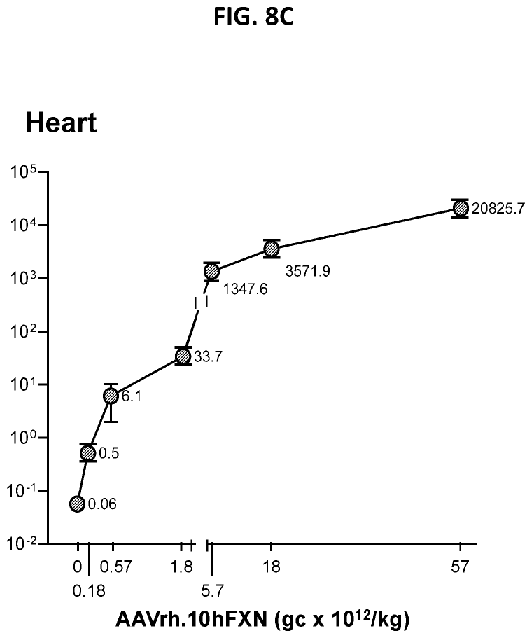

To determine if there was additional benefit from higher doses and higher frataxin expression levels, we intravenously administered to 7-week-old MCK mice doses of AAVrh.10FXN increasing by half-log increments of vehicle (0 gc/kg), 1.8×10¹¹ gc/kg, 5.7×10¹¹ gc/kg, 1.8×10¹² gc/kg, 5.7×10¹² gc/kg (3.3×), 1.8×10¹³ gc/kg (10×) and 5.7×10¹³ gc/kg (33×) (FIG. 8A). With the reference of the original dose based on heterozygous cardiac FXN level as target (1.8×10¹² gc/kg), these doses are referred to these as 3.3×, 10× and 33×) dose cohorts. Following vector administration, health and behavioral assessments, echocardiograms, and cardiac and liver human FXN levels were obtained (FIG. 8B and FIG. 8C). All of the higher dose cohorts had human cardiac FXN levels in excess of the target based on non-FA human heart rising to 20,800±3,200 ng/mg protein in the 33× dose cohort (FIG. 8C).

### Cardiac Function as Assessed by Echocardiography

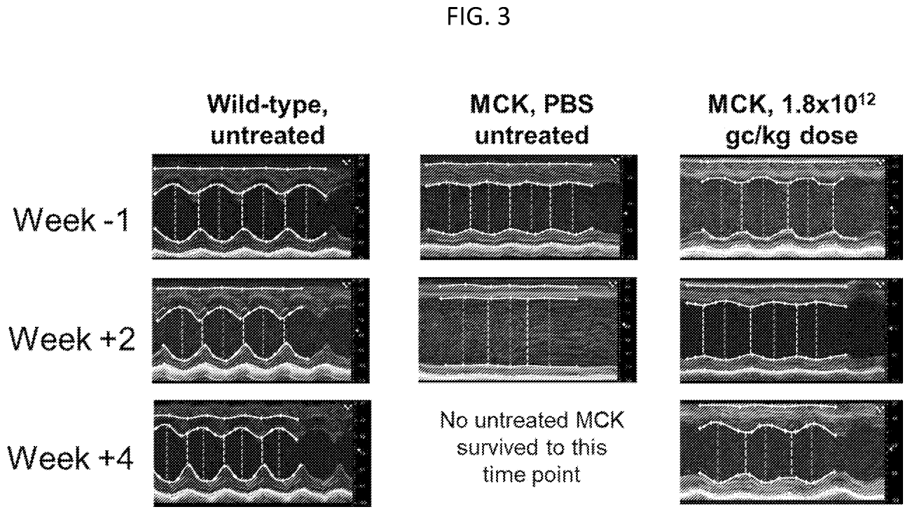

Cardiac function in mice administered 1.8×10¹² gc/kg dose of AAVrh.10hFXN was assessed by echocardiography and compared to untreated, WT MCK mice and MCK mice mock-treated with PBS. FIG. 3 shows a series of echocardiography images comparing three treatments. Echocardiography images of the left ventricle, parasternal short axis view were taken one-week prior to administration, two-weeks after administration and 4-weeks after administration. Images were used to assess the lateral ventricular wall and the septal wall of the left ventricle and their relative positioning during systole and diastole. No untreated MCK mice that were administered PBS mock-control survived to the four-week time point.

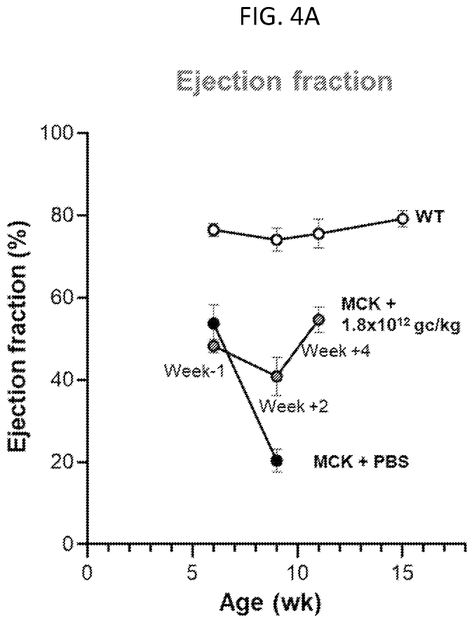

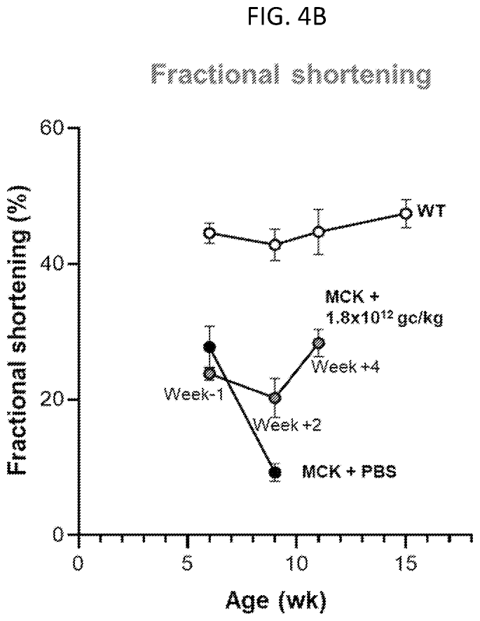

The cardiac ejection fraction and fractional shortening were measured over the course of the experiment. FIGS. 4A-B show the ejection fraction and fractional shortening of mice in the three treatments, respectively. Administration of a 1.8×10¹² gc/kg dose improved the ejection fraction and fractional shortening (p<0.05, both comparisons) compared to PBS controls.

Assessed by echocardiogram, compared to the reference dose (1.8×10¹² gc/kg), there was a significant, dose-dependent increase in ejection fraction at 11 weeks from 18.9±4.5% in the untreated control to 31.9±2.9% in the reference dose cohort, increasing to 54.5±9.8% in the 10× (1.8×10¹³ gc/kg) dose cohort. At the 33× dose (5.7×10¹³ gc/kg), there was a decrease in ejection fraction, consistent with cardiac toxicity. In summary, there is a substantial survival and cardiac benefit in AAV-mediated expression of frataxin above the normal physiological level in the MCK mouse model of Friedrich's ataxia, but toxicity at high doses.

### Survival

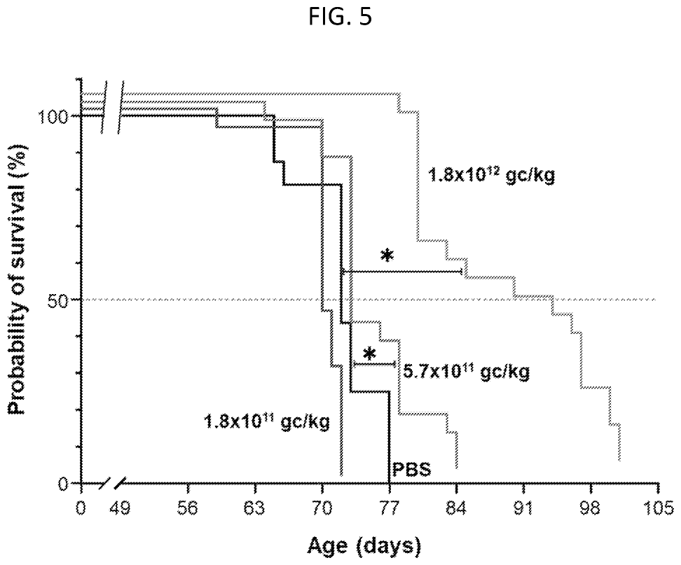

The survival of mice administered three doses of AAVrh.10hFXN was assessed and compared to mock-treated mice administered with PBS. Survival curves were generated to compare percent survival of mice over time following each treatment. A comparison of survival curves (Kaplan-Meier) between PBS control treated cohort vs AAVrh.10hFXN dose cohorts was done using the Log-rank (Mantel-Cox) test. As shown in FIG. 5, a small, but significant improvement in mortality was seen with 5.7×10¹¹ gc/kg dose (p<0.01, compared to control), the minimal effective dose. The 1.8×10¹² gc/kg significantly effective dose-mediated a 21.5% improvement in mortality (p<0.001 compared to untreated controls).

There was an increase in survival increasing from a median of 87.5 days at the reference dose (1.8×10¹² gc/kg) to 119 days in the 3.3× cohort (5.7×10¹² gc/kg), plateauing at 128 days in the 10× (1.8×10¹³ gc/kg) cohort. At the 33× dose (5.7×10¹³ gc/kg), there was a varied impact on survival, with some animals surviving over 200 days but with some deaths earlier than those in the 10× cohort, likely representing toxicity at this dose.

### Conclusion

In MCK mice, a trend of mild improvement was seen at 5.7×10¹¹ gc/kg, the minimum effective dose, on survival; this dose produced 26% of normal human endogenous levels (when combined with the 16% residual levels in FA subjects) and showed a 4% improvement in mortality. Significant improvements were seen at 1.8×10¹² gc/kg for body weight, cardiac function and survival; this dose generated 73% of normal human endogenous levels (when combined with the 16% residual levels in FA subjects) in MCK mice and led to a beneficial outcome with significant improvement in ejection fraction and fractional shortening compared to untreated MCK mice. These data identify a minimum and a significantly effective dose that have a potential to be clinically relevant for the treatment of the cardiac manifestations of Friedreich's ataxia.

### Toxicology Study in Mice

Seven-week-old WT C57Bl/6N mice were administered 1.2×10¹², 3.7E×10¹², and 1.2×10¹³ gc/kg LX2006 (AAVrh.10hFXN i.e. AAV vector encoding frataxin) for 1 and 3 months, and 5.6×10¹¹, 1.8×10¹², and 5.6×10¹² gc/kg LX2006 for 10 months to be evaluated for safety and toxicity.

Animals in the 1- and 3-month cohorts survived to their scheduled terminations while those in the 10-month cohort had two time periods of body weight loss thus necessitating early euthanasia. The first timepoint was approximately 1.5 months (6 weeks) for 4/12 males in the 5.6×10¹² gc/kg 10-month cohort. The early deaths were attributed to liver toxicity in the 10-month cohort. Though the human frataxin protein (hFXN) levels were not determined in these mice, based on a similar dose group (3.7×10¹² gc/kg) from the 1-month cohort, the liver hFXN levels were found to be high and could have contributed to the toxicity. A collection of less severe (but still adverse) hepatic findings were seen at the 1- and 3-month timepoint in the mid (3.7×10¹² gc/kg) and high (1.2×10¹³ gc/kg) dose males with no adverse effects in females up to 3 months postdose.

The second timepoint of weight loss that led to early euthanasia in the 10-month cohort occurred at 7 months (30 weeks) postdose. The remaining animals in the high dose (5.6×10¹² gc/kg) group and 6/12 female mice in the mid dose (1.8×10¹² gc/kg) group had minimal to moderate hepatic changes which were similar to those in the scheduled 3-month cohort. Clinical pathology changes were limited to aspartate aminotransferase (AST) and creatine kinase (CK) elevations in the 5.6×10¹² gc/kg group. By this time point, the high hepatic hFXN levels that were seen in 1 month had decreased while cardiac hFXN levels had increased such that levels were comparable in the 5.6×10¹² gc/kg group. A decrease in the cardiac succinate dehydrogenase (SDH) histochemistry score in male mice (88% of the male control value) at this dose level supports the hypothesis that both hepatic and possibly cardiac frataxin levels may have contributed to the weight loss in the mice that were euthanized at 7 months.

The only adverse treatment-related finding at the scheduled 10-month necropsy was hepatocellular carcinoma (HCC), which was observed in 1/6 males at 5.6×10¹¹ gc/kg and in 3/6 males at 1.8×10¹² gc/kg. While this finding was considered adverse in the context of the study, a large body of available data suggests that HCC observed in mice after AAV treatment is unlikely to translate to risks for humans, as it has not been observed in higher species or humans (FDA, Toxicity Risks of Adeno-associated Virus Vectors for Gene Therapy (GT)” 2-3 September, 2021). Thus, clinical subjects are highly unlikely to be at risk of developing HCC following AAVrh.10hFXN treatment.

Ten months post-treatment, the lowest dose level of 5.6×10¹¹ gc/kg, there were no treatment-related clinical signs, body weight changes, hematologic or clinical chemistry findings, including changes in serum troponin, and no histopathology findings except for tracheobronchial lymph node follicular hyperplasia (indicative of an immunologic response, which is not considered adverse) and HCC in one male mouse. No comparable observations of AAV-related hepatocellular neoplasms have been observed in species other than mice, either animals in AAV preclinical studies or human subjects in AAV clinical trials (FDA 2021), so clinical subjects are highly unlikely to be at risk of developing HCC following AAVrh.10hFXN treatment.

Central nervous system (CNS) pathology (spinal cord and brain) in mice was unremarkable at all doses and all time points. No dorsal root ganglion (DRG) toxicity was observed at any doses in any animal.

IV administration of AAVrh.10hFXN to male and female C57Bl/6 mice resulted in dose- and time-dependent toxicity that appeared to correlate with levels of hFXN expression in liver and possibly heart (based on cardiac SDH histochemistry reductions, relative to control, at the 5.6×10¹² gc/kg dose level in male mice euthanized at Week 6 and Week 30). At the low dose of 5.6×10¹¹ gc/kg there were no adverse findings throughout the study but one male mouse at the 10-month necropsy had an HCC. HCC were also observed in 3/6 male mice at the 1.8×10¹² gc/kg dose level (the only other dose level where male animals survived for the entire study), suggesting that this is a treatment-related event. This suggests that the 5.6×10¹¹ gc/kg dose level represents the lowest-observed-adverse-effect-level (LOAEL) in male mice and a no-observed-adverse-effect level (NOAEL) in female mice. However, AAV-related HCC in male mice are not expected to be relevant to humans, based on the lack of corresponding findings in other species. While this finding was considered adverse in the context of the study, available data suggests HCC observed in mice after AAV treatment is unlikely to translate to risks for humans, and has not been observed in higher species or humans (FDA 2021).

### Non-Human Primate Dosage Study

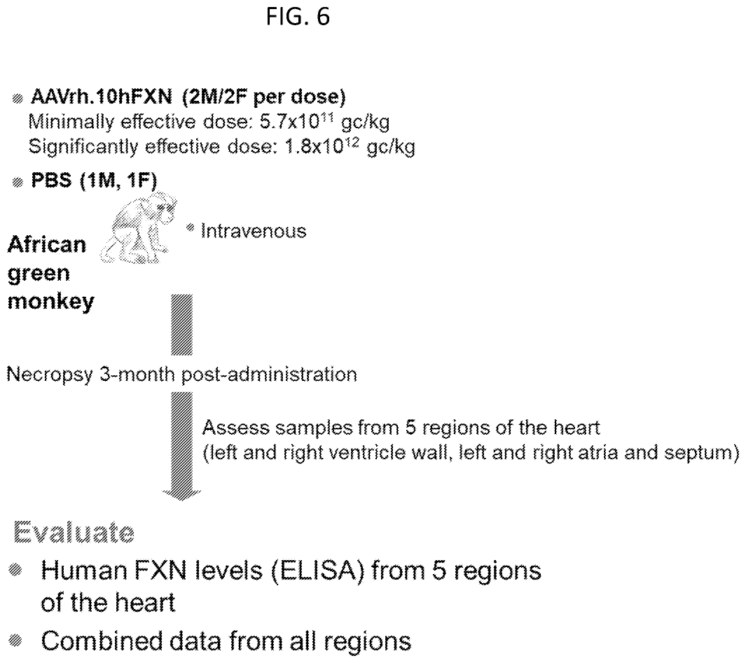

To determine if the significantly effective dose of 1.8×10¹² gc/kg that was determined in MCK mice could achieve heterozygote levels in a large animal model, this dose was administered intravenously to African Green nonhuman primates (NHPs) (n=10). FIG. 6 shows a schematic depicting the method of administration and assessment of dosages in NHPs. After 12 weeks following administration of the significantly effective dose, the levels in the heart were ˜18 ng/mg, comparable to levels in the range estimated necessary to convert the FA homozygote to an FA heterozygote.

Two doses, comprising the minimal effective dose (5.7×10¹¹ gc/kg) and the significantly effective dose (1.8×10¹² gc/kg) as determined in MCK mice were assessed in non-human primates. Two doses of AAVrh.10hFXN were administered intravenously to African Green nonhuman primates (4 primates per dose; 2/sex/dose) and compared to PBS administration (2 primates total; 1/sex). After 12 weeks post-administration, samples from five regions of the heart (left/right ventricle wall, left/right atria, and septum) were collected and examined for hFXN levels. The data from all five regions was combined for total cardiac tissue measurement.

Expression of hFXN in Nonhuman Primate Cardiac Tissue

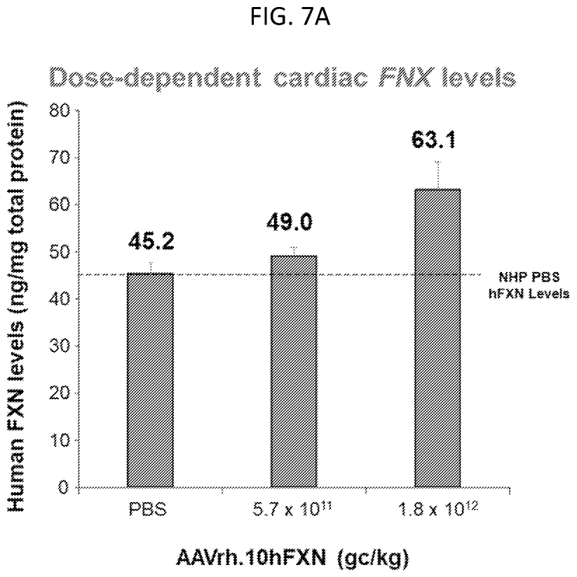

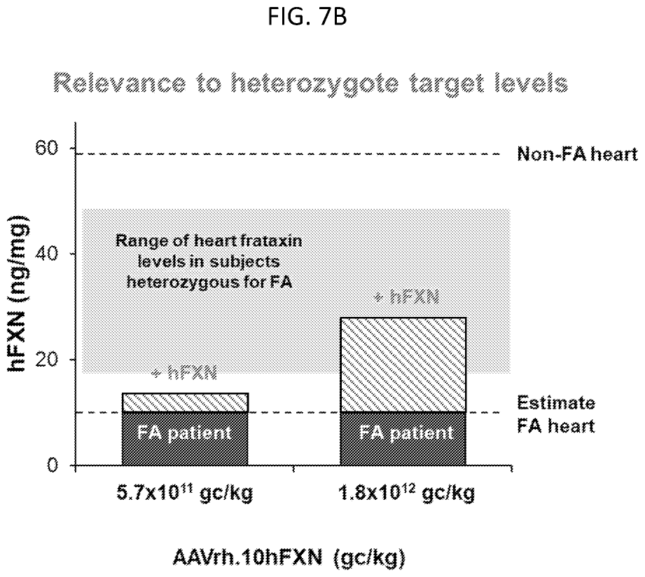

Assessment of hFXN total protein in cardiac tissue of NHPs following administration of two dosages of AAVrh.10hFXN showed a dose-dependent increase in cardiac hFXN expression. The heart samples for NHP administered PBS had background levels of FXN due to the cross reactivity of the antibody. As shown in FIG. 7A, intravenous administration of 5.7×10¹¹ gc/kg and 1.8×10¹² gc/kg of AAVrh.10hFXN were associated with cardiac hFXN protein levels of 49 ng/mg and 63.1 ng/mg, respectively. The PBS control group had hFXN protein levels of 45.2 ng/mg.

To establish if the cardiac levels observed in NHPs are in the “therapeutic” range, these hFXN levels were added to the anticipated endogenous FXN levels as previously cited above. As shown in FIG. 7B, at 5.7×10¹¹ and 1.8×10¹² gc/kg, the increase in FXN levels when added to endogenous reached 22% and 46% of normal human heart FXN levels, respectively, and within the heterozygous range at the 1.8×10¹² gc/kg dose.

### Conclusion

In nonhuman primates, intravenous doses of 5.7×10¹¹ and 1.8×10¹² gc/kg, generated 22 and 46%, respectively, of normal endogenous levels when combined with the 16% residual levels in FA subjects.

A careful determination of the required levels of the therapeutic FXN protein in humans set the threshold for “non-toxic” levels. Dose titration studies in two animal models (MCK mice and nonhuman primates) demonstrated the potential of doses of AAVrh.10hFXN (1.8×10¹¹, 5.6-5.7×10¹¹ and 1.8×10¹² gc/kg) in mediating meaningful improvements in FA disease progression. Together these data identify a therapeutically effective dose that has a potential to be clinically relevant for the treatment of the cardiac manifestations of Friedreich's ataxia.

### Example 2: Assessment of Human Dosage for Frataxin AAV Vector Therapy

A first-in-human (FIH), 52-week, dose-ascending, open-label study of LX2006 (frataxin AAV vector of the disclosure) will be conducted in participants who have FA with evidence of cardiomyopathy, followed by a long-term follow-up (LTFU) portion of the study for all participants who receive LX2006.

Two sequential cohorts (N=5 adult FA participants in each) will be enrolled at escalating doses of LX2006 (1.8×10¹¹, 5.6×10¹¹ gc/kg). All participants will remain in the hospital for two nights after dosing and will then be discharged on Day 3. The first two participants of each cohort will reside near the site during the first 4 weeks. The next 3 participants (#3, 4, and 5) will stay near the site for a minimum of 2 weeks. There will be at least a 4-week interval between participants in each cohort in order to obtain the 4-week safety data (including laboratory tests) from the previous participant. Available safety data from all previous participants will also determine the dosing of additional participants.

The treatment duration will be a single administration of LX2006, at the appropriate dose depending on study cohort, administered as a slow intravenous (IV) infusion over 60 minutes. Further, patients will be administered prophylactic prednisone to minimize host immune response to AAV-based therapy. Participants receive 40 mg once daily (QD) from Day-1 through Week 8. Tapering begins on Week 9 and continues through Week 14 but dose adjustments are made depending on alanine aminotransferase (ALT) and aspartate aminotransferase (AST) values, as described below. Prednisone will be taken QD and recommended to be taken in the morning.

### Safety and Efficacy Assessment

Since hFXN expression is the key determinant associated with safety and efficacy in FA disease, the nonclinical studies provide a better understanding of which dose was associated with toxicity and which doses could yield FXN expression that are both safe and efficacious. In summary, it is anticipated that achieving approximately 30% of normal human myocardial FXN levels would provide a clinically meaningful benefit in FA patients.

### Primary Efficacy Objective

Assess the effect on LX2006 on peak V02 based on Cardiopulmonary exercise testing (CPET) (using arm ergometry). Measurement will look at change in peak VO2 at week 52 versus pre-treatment baseline.

### Secondary Objectives

To assess the effect of LX2006 on Left Ventricular Mass index (LVMi) measured by cardiac MRI. Change from baseline in LVMi will be measured at Week 52. Change from baseline in LVMi will be measured at Weeks 12, 24, and 36 and annually during the 4 years in the LTFU.

To assess the effect of LX2006 on peak VO2 based on CPET (using arm ergometry). Change from baseline in peak VO2 will be measured at Weeks 12, 24, 36, and annually during the 4 years in the LTFU, for a total of 5 years postdose.

To measure global longitudinal strain based on cardiac Magnetic resonance imaging (MRI). Change from baseline in global longitudinal strain will be measured at Weeks 12, 36, 24, 36, and 52, and annually in the LTFU for 4 years, for a total of 5 years postdose.

To measure stroke volume based on cardiac MRI. Change from baseline in stroke volume will be measured at Weeks 12, 24, 36, and 52, and annually in the LTFU for 4 years, for a total of 5 years postdose.

To measure Left ventricular ejection fraction (LVEF) based on cardiac MRI. Change from baseline in LVEF will be measured at Weeks 12, 24, 36, and 52, and annually in the LTFU for 4 years, for a total of 5 years postdose.

To measure cardiac fibrosis based on cardiac MRI. Change from baseline in cardiac fibrosis will be measured at Weeks 12, 24, 36, and 52, and annually in the LTFU for 4 years, for a total of 5 years postdose.

To measure serum NTproBNP, hsTNT, and CK-MB levels. Change from baseline in cardiac biomarkers will be measured at Weeks 2, 12, 24, 36, and 52, and annually in the LTFU for 4 years, for a total of 5 years postdose.

To measure cardiac arrythmias based on remote cardiac rhythm monitoring. Presence and severity of cardiac arrythmias at Weeks 12, 24, 36, and 52, and annually in the LTFU for 4 years, for a total of 5 years postdose.

To measure fatigue based on the Modified Fatigue Impact Scale (MFIS) and the Fatigue Severity Scale (FSS). Presence and severity of fatigue will be measured at Weeks 12, 24, 36, and 52, and annually in the LTFU for 4 years, for a total of 5 years postdose.

To measure exertional symptoms based on the Modified Borg Dyspnea, Borg Rating of Perceived Exhaustion (RPE), and angina scale during CPET. Change from baseline of the Modified Borg Dyspnea, Borg RPE, and angina scale will be measured at Weeks 12, 24, 36, and 52, and annually in the LTFU for 4 years, for a total of 5 years postdose.

### Tertiary Objectives

To determine the vector copy number (VCN) in cardiac biopsies. Change from baseline in vector copies will be measured at Week 12.

To measure cardiac parameters based on echocardiogram (ECHO), including but not limited to LVMi, LVEF, and global longitudinal strain. Change from baseline in cardiac ECHO measurements will be measured at Weeks 1, 2, 4, 12, 24, 36, and 52, and annually in the LTFU for 4 years, for a total of 5 years postdose.

To measure 12-lead electrocardiogram (ECG) parameters, including but not limited to ST-T wave changes. Change from baseline in 12-lead ECG will be measured at Weeks 1, 2, 4, 12, 24, 36, and 52, and annually in the LTFU for 4 years, for a total of 5 years postdose.

To measure symptoms based on the Seattle Angina Questionnaire, the Shortness of Breath-Daily Activities score, visual edema scores, and ankle diameter. Change from baseline in the Seattle Angina Questionnaire, the Shortness of Breath-Daily Activities score, will be measured at Weeks 12, 24, 36, and 52, and annually in the LTFU for 4 years, for a total of 5 years postdose.

To assess the effect of LX2006 on other CPET measures, including, but not limited to: 1. Minute ventilation to carbon dioxide production (VE/VCO2) slope; 2. Partial pressure of end-tidal CO2 (PETCO2): 3. Duration of exercise during CPET; 4. VO2 at ventilatory threshold (VT); 5. Oxygen uptake efficiency slope (OUES). Change from baseline in VE/VCO2 slope, PETCO2, duration of exercise, VO2 at VT, and OUES will be measured at Weeks 12, 24, 36, and 52, and annually in the LTFU for 4 years, for a total of 5 years postdose.

### Exploratory Objectives

To explore cardiac extracellular volume (ECV). Change from baseline in cardiac ECV will be measured at Weeks 12, 24, 36, and 52, and annually in the LTFU for 4 years, for a total of 5 years postdose based on cardiac MRI.

To explore the effect of LX2006 on FXN protein levels in buccal cells and PBMCs. Change from baseline of FXN protein in buccal cells and PBMCs will be measured at Weeks 4, 12, 24, 36, and 52.

To explore the effect of LX2006 on measures of FA, including but not limited to Friedreich's Ataxia Rating Scale (FARS), Friedreich's Ataxia Rating Scale-Activities of daily living (FARS-ADL), modified Friedreich's Ataxia Rating Scale (mFARS), Scale for Assessment and Rating of Ataxia (SARA). Change from baseline of FARS, FARS-ADL, mFARS, SARA, and others will be measured at Weeks 12, 24, 36, and 52, and annually in the LTFU for 4 years, for a total of 5 years postdose.

To explore the vector shedding of LX2006. Measurement of vector shedding in buccal, urine, blood, and stool samples will be taken at Weeks 1, 2, 4, 12, 24, and 52, and annually in the LTFU for 4 years, up to 5 years postdose.

### Cardiac Biopsy:

Fluoroscopy and ultrasound will be used in the cardiac biopsies which will be performed before initiation of gene therapy and 12 weeks post-treatment by appropriately trained staff at the study site. All subjects will have a physical exam to determine if there is any intercurrent condition that places a participant at higher risk. The collected pieces of the septum may be divided up to approximately 8 pieces. Assessments will include vector copy number, FXN protein, cardiomyocyte FXN staining (if available), and electron microscopy.

Details for tissue sampling will be provided in a Cardiac Biopsy Manual. In addition, during the cardiac biopsy procedure, a Swan-Ganz catheter will be used to measure cardiac output using Fick's formula. All tissues will be analyzed together after the final patient in each cohort has the biopsy at Week 12.

### Cardiac ECHO:

All participants undergo cardiac ECHO at Screening/Baseline and timepoints detailed in the disclosure, which includes measurements of LVMi, LVEF, stroke volume, and strain.

### Cardiac MRI:

All participants undergo MRI scans of the heart at Screening/Baseline and timepoints detailed in the disclosure. In the LTFU, cardiac MRI scans will be performed at Day 1 (if not already done during the Week 52 visit during dose-escalation) and annually. Participants must be able to tolerate contrast (e.g., gadolinium), as noted in Exclusion Criteria #13. Cardiac MRI assessments include measurements of LVMi, LVEF, stroke volume, strain, and fibrosis.

### Cardiac Rhythm Monitoring:

Measurement of cardiac arrythmias is based on remote cardiac rhythm monitoring. A patch is placed on the participant's chest and worn remotely for 7 consecutive days to be assessed centrally following in-person visits.

### Clinician Reported Outcomes

Friedreich's Ataxia Rating Scale (FARS) and mFARS

The FARS is a clinician-reported outcome measure consisting of three subscales: a general score for ataxia, a score for activities of daily living (ADL) and a neurological examination. The neurological exam is an instrument developed to measure neurological function in individuals with FA. The full instrument has 25 items with a maximum total score of 125. The five domains of the FARS are bulbar function, upper limb coordination, lower limb coordination, peripheral nervous system, and upright stability. Each item is to be rated based on the status of the subject during the examination.

The modified FARS (mFARS) is a subset of the neurological exam in the FARS scale. The mFARS score is derived from the completed neurological exam for the FARS scale. The mFARS has four of the five domains of the FARS assessment; it does not include the peripheral nervous system.

### Scale for Assessment and Rating of Ataxia (SARA)

The SARA is an 8-item performance scale that assesses gait, stance, sitting, speech disturbance, finger chase, nose-finger test, fast alternating hand movements, and heel shin slide. The total score may range from 0 indicating no ataxia to 40 indicating the most severe ataxia.

### Patient Reported Outcomes

### Shortness of Breath-Daily Activities Score (SOBDA)

The SOBDA 13-item questionnaire is a daily questionnaire developed to quantify a participant's perception of dyspnea related to daily activities and to assess changes over time. Participants will be asked to complete the SOBDA daily during the 7 days prior to travel for the in-person visits.

### Seattle Angina Questionnaire (SAQ)

The SAQ is a widely used patient-reported outcomes measure in patients with heart disease. The SAQ is self-administered and consists of 19-items. The questionnaire measures the following 5 domains of health-related quality of life with a recall period of 4 weeks. These domains are: physical limitation (9 items), angina stability (1 item), angina frequency (2 items), treatment satisfaction (4 items), and disease perception (3 items). The items are scored on a 5- or 6-point Likert scale.

### Fatigue Scales (FSS, MFIS)

The Fatigue Severity Scale (FISS) is a patient-reported scale assessing the impact of fatigue. A 7-point Likert scale is used to endorse or deny 9-items. The recall period for this instrument is within the last week. The Visual Analogue Fatigue Scale at the end of the instrument captures global fatigue with 0 indicating the worst level of fatigue and 10 representing normal.

The Modified form of the Fatigue Impact Scale (MFIS) assesses the effects of fatigue in terms of physical, cognitive, and psychosocial functioning. The instrument contains 21 items and has a recall period of 4 weeks.

### Pittsburgh Sleep Quality Index (PSQI)

The PSQI is a 19-item patient reported instrument used to measure sleep quality and disturbances over the past 4 weeks. The 7 components of sleep quality are 1) sleep duration, 2) sleep disturbance, 3) sleep latency, 4) daytime dysfunction due to sleepiness, 5) sleep efficiency, 6) overall sleep quality, and 7) sleep medication use.

### Short Form Health Survey (SF-36)

The SF-36 is a 36-item patient reported instrument to measure health-related quality of life. It measures physical functioning, role physical, bodily pain, general health, vitality, social functioning, role emotional, and mental health.

### Assessments During CPET

### Modified Borg Dyspnea Scale

The Modified Borg Dyspnea Scale is a 10-point scale used to measure difficulty in breathing as reported by the patient during CPET, with 10 representing the maximum difficulty and 0 representing no dyspnea at all.

### Angina Scale

The Angina Scale, a 4-point scale with verbal descriptors, is used to standardize perceived chest pain, as reported by the participant during CPET.

### Borg Rating Scale of Perceived Exertion (Borg RPE)

The Borg Scale is a tool for measuring an individual's perceived effort and exertion during physical work. It is a 15-point scale with verbal descriptors to standardize perceived exertion, as reported by the participant during CPET.

### Biomarker Assessment

Blood samples are collected for cardiac biomarkers (e.g., FXN expression, Serum N-terminal-pro hormone B-type Natriuretic Peptide [NTproBNP], hsTNT, creatine kinase (CK) and its MB isoenzyme [CK-MB]) that may be informative for this therapeutic approach. Samples will be collected according to the schedule described in the SoA and as detailed in a Laboratory Manual provided separately to sites. Biomarker samples should be collected prior to the CPET assessment.

We claim:

1. A method of treating or preventing cardiomyopathy associated with Friedreich ataxia in a subject, the method comprising administering to the subject a therapeutically effective amount of an adeno associated virus (AAV) vector which comprises a nucleic acid sequence encoding a frataxin (FXN) polypeptide or a fragment thereof, wherein the AAV vector comprises in the 5′ to 3′ direction:
a first AAV ITR sequence;
an enhancer sequence;
a promoter sequence;
a chimeric intron;
the nucleic acid sequence encoding a frataxin (FXN) polypeptide;
a polyA sequence; and
a second ITR sequence, and
wherein the vector is administered to the subject intravenously at a dose ranging from about 1.0×10¹⁰ gc/kg to about 6.0×10¹⁴ gc/kg.

2. A method of treating or preventing cardiomyopathy associated with Friedreich ataxia in a subject, the method comprising administering to the subject a therapeutically effective amount of an adeno associated virus (AAV) vector which comprises a nucleic acid sequence encoding a frataxin (FXN) polypeptide or fragment thereof, wherein the AAV vector comprises the nucleic acid sequence set forth in SEQ ID NO: 9, wherein the vector is administered the subject intravenously at a dose of about 1.8×10¹¹ gc/kg or about 5.6×10¹¹ gc/kg.

3. The method of any one of the preceding claims, wherein the nucleic acid sequence encoding a frataxin (FXN) polypeptide comprises the nucleic acid sequence set forth in SEQ ID NO: 3.

4. The method of any one of the preceding claims, wherein the first ITR sequence comprises the nucleic acid sequence set forth in SEQ ID NO: 1.

5. The method of any one of the preceding claims, wherein the second ITR sequence comprises the nucleic acid sequence set forth in SEQ ID NO: 2.

6. The method of any one of the preceding claims, wherein the enhancer sequence comprises the nucleic acid sequence set forth in SEQ ID NO: 5.

7. The method of any one of the preceding claims, wherein the promoter sequence comprises the nucleic acid sequence set forth in SEQ ID NO: 6.

8. The method of any one of the preceding claims, wherein the polyA sequence comprises the nucleic acid sequence set forth in SEQ ID NO: 8.

9. The method of any one of the preceding claims, wherein the AAV vector comprises the nucleic acid sequence set forth in SEQ ID NO: 9.

10. The method of any one of the preceding claims, wherein the AAV vector is packaged as an AAV viral vector comprising an AAV capsid protein.

11. The method of any one of the preceding claims, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

12. The method of claim 11, wherein the AAV capsid protein is an AAVrh10 capsid protein.

13. The method of any one of the preceding claims, wherein the dose is about 1.8×10¹¹ gc/kg.

14. The method of any one of the preceding claims, wherein the dose is about 5.6×10¹¹ gc/kg.

15. The method of any one of the preceding claims, wherein the subject is further administered prednisone.

16. The method of claim 15, wherein the prednisone is administered at a dosage of:
40 mg, once daily 24 hours prior to AAV viral vector administration;
40 mg once daily for week 1 through week 8 post-AAV viral vector administration;
30 mg once daily for week 9 post-AAV viral vector administration;
20 mg once daily for week 10 post-AAV viral vector administration;
10 mg once daily for week 11 post-AAV viral vector administration;
5 mg once daily for week 12 post-AAV viral vector administration;
2.5 mg once daily for week 13 post-AAV viral vector administration; and
2.5 mg every other day for week 14 post-AAV viral vector administration.

17. The method of any one of the preceding claims, wherein the intravenous administration occurs over about 60 minutes.

18. The method of any one of the preceding claims, wherein the subject experiences an increase in peak VO2 following AAV vector administration relative to a pre-AAV vector administration baseline.

19. The method of claim 18, wherein the peak VO2 in the subject is measured about, 12 weeks, about 24 weeks, about 36 weeks, about 52 weeks, about 18 months, about 2 years, about 3 years, about 4 years, and/or about 5 years post-AAV vector administration.

20. The method of claim 18 or 19, wherein peak VO2 is measured by cardiopulmonary exercise testing (CPET) using arm ergometry.

21. The method of any one of the preceding claims, wherein the subject experiences a decrease in Left Ventricular Mass index (LVMi) following AAV vector administration relative to a pre-AAV vector administration baseline.

22. The method of claim 21, wherein the decrease in LVMi is measured about 12 weeks, about 24 weeks, about 36 weeks, about 52 weeks post-AAV vector administration, about 18 months, about 2 years, about 3 years, about 4 years, and/or about 5 years post-AAV vector administration.

23. The method of claim 21 or 22, wherein the decrease in LVMi is measured by cardiac MRI.

24. The method of any one of the preceding claims, wherein the subject, following AAV vector administration, experiences one or more of decreased global longitudinal strain, increased stroke volume, increased left ventricular ejection fraction (LVEF), and decreased or stable cardiac fibrosis as measured by cardiac MRI relative to a pre-AAV vector administration baseline.

25. The method of claim 24, wherein the measurement by cardiac MRI occurs about 12 weeks, about 24 weeks, about 36 weeks, about 52 weeks, about 18 months, about 2 years, about 3 years, about 4 years, and/or about 5 years post-AAV vector administration.

26. The method of any one of the preceding claims, wherein the subject, following AAV vector administration, experiences decreased serum NTproBNP, hsTNT, and CK-MB levels relative to a pre-AAV vector administration baseline.

27. The method of any one of the preceding claims, wherein the subject, following AAV vector administration, experiences decreased cardiac arrythmias relative to a pre-AAV vector administration baseline.

28. The method of claim 27, wherein cardiac arrythmias are evaluated by remote cardiac rhythm monitoring.

29. The method of any one of the preceding claims, wherein the subject, following AAV vector administration, experiences decreased fatigue as measured by the Modified Fatigue Impact Scale (MFIS) or Fatigue Severity Scale (FSS) relative to a pre-AAV vector administration baseline.

30. The method of any one of the preceding claims, wherein the subject, following AAV vector administration, experiences improvements in exertional symptoms (during CPET via arm ergometry) as measured by the Modified Borg Dyspnea, Borg Rating of Perceived Exhaustion (RPE), and the angina scale using CPET relative to a pre-AAV vector administration baseline.

31. The method of any one of the preceding claims, wherein the subject, following AAV vector administration, experiences increased FXN expression levels relative to a pre-AAV vector administration baseline.

32. The method of claim 31, wherein the FXN levels are myocardial FXN expression levels.

33. The method of claim 32, wherein myocardial FXN expression is measured by cardiac biopsy.

34. The method of claim 31, wherein the subject experiences an at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 150%, at least about 200%, at least about 300%, at least about 400% increase in FXN expression levels.

35. The method of any one of the preceding claims, wherein the subject, following AAV vector administration, experiences improvements or stabilizations in the Friedreich's Ataxia Rating Scale (FARS) or modified Friedreich's Ataxia Rating Scale (mFARS) relative to a pre-AAV vector administration baseline.

36. The method of any one of the preceding claims, wherein the subject, following AAV vector administration, experiences improvements or stabilizations in the Scale for Assessment and Rating of Ataxia (SARA) relative to a pre-AAV vector administration baseline.

37. The method of any one of the preceding claims, wherein the subject, following AAV vector administration, experiences improvements or stabilizations in the Shortness of Breath-Daily Activities Score (SOBDA) relative to a pre-AAV vector administration baseline.

38. The method of any one of the preceding claims, wherein the subject, following AAV vector administration, experiences improvements or stabilizations in the Seattle Angina Questionnaire (SAQ) relative to a pre-AAV vector administration baseline.

39. The method of claim 1, wherein the dose is from about 1.0×10¹⁰ gc/kg to about 6.0×10¹² gc/kg.

40. The method of claim 1, wherein the dose is from about 6.0×10¹² gc/kg to about 1.0×10¹⁴ gc/kg.


SEQUENCE LISTING (9 sequences)

SEQ ID NO: 1 - DNA, 141 bp, synthetic construct
  misc_feature 1..141: note = ITR sequence
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 129 bp, synthetic construct
  misc_feature 1..129: note = ITR sequence
  source 1..129: mol_type = other DNA
ggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 3 - DNA, 633 bp, Homo sapiens
  source 1..633: mol_type = other DNA
atgtggactctcgggcgccgcgcagtagccggcctcctggcgtcacccagcccggcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttga

SEQ ID NO: 4 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 5 - DNA, 380 bp, synthetic construct
  misc_feature 1..380: note = CMV enhancer
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 6 - DNA, 278 bp, synthetic construct
  misc_feature 1..278: note = Chicken beta-Actin promoter
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 7 - DNA, 1017 bp, synthetic construct
  misc_feature 1..1017: note = chimeric intron
  source 1..1017: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 8 - DNA, 56 bp, synthetic construct
  misc_feature 1..56: note = b-globin polyA
  source 1..56: mol_type = other DNA
aataaaggaaatttattttcattgcaatagtgtgttggaattttttgtgtctctca

SEQ ID NO: 9 - DNA, 3309 bp, synthetic construct
  misc_feature 1..3309: note = ITR to ITR sequence
  source 1..3309: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcctaggatatcaggagccaccatgtggactctcgggcgccgcgcagtagccggcctcctggcgtcacccagcccggcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgagcaattgttaattaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttatcgataccgtcgacctcgagggggggcccggtacccagcttttgttccctttgcggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcagg